(19) United States
(12) Patent Application Publication
(10) Pub. No.: US 2020/0230177 A1
(43) Pub. Date: Jul. 23, 2020

(54) IMPLANTS, METHODS FOR MAKING IMPLANTS AND METHODS OF TREATING LIPOATROPHY DEFECTS THEREWITH

(71) Applicant: BioTime, Inc., Alameda, CA (US)

(72) Inventors: Oscar Cuzzani, Walnut Creek, CA (US); Tom Zarembinski, Castro Valley, CA (US); Michael Onorato, Alameda, CA (US)

(21) Appl. No.: 16/708,725

(22) Filed: Dec. 10, 2019

Related U.S. Application Data
(63) Continuation of application No. PCT/US2018/037174, filed on Jun. 12, 2018.
(60) Provisional application No. 62/563,041, filed on Sep. 25, 2017.
(60) Provisional application No. 62/519,145, filed on Jun. 13, 2017.
(60) Provisional application No. 62/518,556, filed on Jun. 12, 2017.

(51) Int. Cl. A61K 35/35; A61K 47/69

(52) U.S. Cl. CPC A61K 35/35; A61K 47/6903

(57) ABSTRACT

The technology of this disclosure pertains generally to the correction of tissue volume deficits, and more particularly to the delivery of cells and tissue to treat soft tissue defects or to add volume to soft tissue where desired.

### CROSS-REFERENCE TO RELATED APPLICATIONS

This application claims priority to, and the benefit of U.S. provisional patent application Ser. No. 62/518,556 filed on Jun. 12, 2017; U.S. provisional patent application Ser. No. 62/519,145 filed on Jun. 13, 2017; and U.S. provisional patent application Ser. No. 62/563,041 filed on Sep. 25, 2017, each of which are incorporated herein by reference in their entirety.

### BACKGROUND

The technology of this disclosure pertains generally to the correction of tissue volume deficits, and more particularly to the delivery of cells to treat soft tissue defects or to add volume to soft tissue where desired.

Lipoatrophy, also called lipodystrophy, is a condition which describes a loss of fat tissue in a localized area. Lipoatrophy may be a result of other conditions which lead to a loss of fat tissue in an area.

Facial and body contour deformities caused by lipoatrophy are typically not classified as life threatening but may significantly impact quality of life. These deformities can be secondary to congenital abnormalities, trauma, surgical resection, aging processes, frequent subcutaneous injections, and disease and arise from a missing volume of subcutaneous soft tissue, resulting in a depression in the skin. Today, contour defect reconstruction remains challenging for reconstructive surgeons. Many currently available materials can be implanted to fill a defect, but none adequately replaces the original form or texture nor provides the permanence of the lost adipose tissue.

For the HIV patient, facial lipoatrophy associated with the chronic stage of the disease can be particularly stigmatizing. HIV-related facial lipoatrophy publicly displays the wasting effect of HIV resulting in a negative professional and personal quality of life and is often cited as a reason for non-compliance with or delay of Antiretroviral (ARV) treatment. Defined as a progressive loss of facial fat, mainly due to a decrease in Bichat's fat pad and temporal fat, it causes the appearance of new skin furrows, deepens the intensity of facial expression lines as well as the areas of natural depression on the face and skull, subsequently wrinkling the face. This facial wrinkling ages the individual prematurely and along with a thinning of the skin allowing musculature and vasculature to be easily seen, results in what is commonly known as “the face of AIDS”.

Although the disorders of body fat distribution, including HIV lipoatrophy, are currently thought to be irreversible, surgery is an option to reverse the appearance of lipodystrophy—either atrophy, hypertrophy, or both. Surgical options include surgically placed alloplastic, autologous, cadaveric, or synthetic implants. While these implants provide short-term improvement, as the facial lipoatrophy (FL) progresses the implant edges become apparent and they often have a rigid feel. In addition, surgical options have a considerable down time and relatively high cost.

In spite of the growing popularity of dermal fillers in resolving lipoatrophies, it has been found that there is little application outside of temporary resolution of facial wrinkles and volumizing the lips and cheeks. As a result, these products are typically useful in small volume (<3 cc) single treatment formats. At present, there are two medical device products approved in the EU and US for subcutaneous injection in the affected facial areas and both cause a fibrotic reaction that is intended to fill the subcutaneous defect caused by the lipoatrophy. A typical treatment regimen involves multiple injections over several months. With the larger volumes needed to correct large defects such as those caused by HIV-related facial lipoatrophies and other caused lipoatrophies, the cost can become prohibitive and the outcomes are variable.

There is a need for an effective solution to treat large and mid-face volume deficiencies no matter the cause of the lipoatrophy or to add volume to soft tissue where desired.

### BRIEF SUMMARY

In certain embodiments, the disclosure provides compositions and methods for restoring and regenerating lost soft tissue in a subject using resorbable matrices to deliver cells and or tissue to lipoatrophic areas.

Accordingly, methods are provided, for slowing the progression of lipoatrophy, slowing the progression of facial lipoatrophy, preventing facial lipoatrophy, preventing facial volume decrease, restoring facial volume, increasing facial volume for greater than 6 months, or treating subcutaneous facial lipoatrophy defects in a subject, by administering a lipo-restoring composition to the face that includes a combination of a resorbable matrix and adipose derived cells, according to certain embodiments.

In other embodiments, the lipoatrophy can be a mid-face volume deficit, a mild to severe submalar volume deficit, a mild to severe perioral volume deficit, or subcutaneous contour defects.

In certain embodiments, the defects can arise from HIV infection or Highly Active Antiretroviral Therapy (HAART).

In other embodiments, the defects may arise from secondary to congenital abnormalities, trauma, surgical resection, the aging process, and disease.

In other embodiments, the defects may arise from infection, diabetes, auto immune disease, acquired generalized lipodystrophy (AGL), Lawrence syndrome, acquired partial lipodystrophy (APL), progressive lipodystrophy, Barraquer-Simons syndrome, injury, weight loss, repeated injection site, or localized pressure.

In certain embodiments, the infection may be caused by one or more of measles, pneumonia, infectious mononucleosis, or hepatitis.

In certain embodiments, the administered adipose derived cells attach, proliferate and differentiate into adipocytes.

In certain embodiments, the adipose derived cells include one or more of: autologous adipose derived cell, stromal vascular cells, stromal vascular fraction, multipotent stem cells, pre-adipocytes, and endothelial precursor cells.

In certain embodiments, the adipose derived cells are isolated from fat tissue harvested from a subject.

In some embodiments, the subject is the same subject that receives the lipo-restoring composition or is an unrelated subject.

In other embodiments, the fat cells are obtained by liposuction.

In some embodiments, about 50-500 mL of dry adipose can be collected from the liposuction.

In other embodiments, the adipose is washed and processed to obtain a cell pellet.

In certain embodiments, a subject's facial volume is measured against a baseline.

In certain embodiments, the baseline includes a measurement made prior to administration.

In certain embodiments, the facial volume change is for greater than 9 months.

In certain embodiments, the facial volume change is for greater than 12 months.

In other embodiments, the facial volume change is for greater than 18 months.

In other embodiments, the resorbable matrix comprises a hydrogel.

In other embodiments, the hydrogel includes thiol-modified hyaluronan, thiol-modified gelatin, and polyethylene glycol diacrylate (PEGDA). In one embodiment, the thiol-modified hyaluronan may be any hyaluronic acid that has a thiol modification that is known in the art, for example, those described in U.S. Pat. Nos. 8,664,788, 6,620,927, and those described in Chemistry and Biology of Hyaluronan, pp. 475-504, December 2004. In one embodiment, the thiol-modified gelatin may be a thiol modified collagen or gelatin molecule.

In certain embodiments, the hydrogel is made by reconstituting the thiol-modified hyaluronan, thiol-modified gelatin, and polyethylene glycol diacrylate (PEGDA) and mixing the thiol-modified hyaluronan, thiol-modified gelatin, and polyethylene glycol diacrylate (PEGDA) together.

In certain embodiments, the hydrogel is made by contacting a first thiolated monomer with GSSG, allowing the first thiolated monomer and the GSSG to react, and adding a second thiolated monomer to the reaction of the second step, thereby forming a hydrogel including the first and second thiolated monomers, but not including glutathione or GSSG.

In certain embodiments, the first thiolated monomer is thiolated carboxymethylated hyaluronan and wherein the second thiolated monomer is thiolated gelatin.

In other embodiments, the resorbable matrix includes an SLF.

In other embodiments, SLF is made by thawing a combination of thiol-modified hyaluronan and thiol-modified gelatin at a temperature of approximately 20° C. or greater; and adding polyethylene glycol diacrylate (PEGDA) to the thawed combination of thiol-modified hyaluronan and thiol-modified gelatin.

In some embodiments, the SLF resorbable matrix composition may be stored from between −80 degrees C. to about 45 degrees C., or from between −20 degrees C. to about 25 degrees C., from between −10 degrees C. to about 4 degrees C., or from between 0 degrees C. to about 10 degrees C.

In other embodiments, the resorbable matrix includes an SLF composition with non-thiol-modified polysaccharides. In other embodiments, the resorbable matrix includes an SLF composition with non-thiol-modified collagen or gelatin, for example. In yet other embodiments, the resorbable matrix includes an SLF composition with both non-thiol-modified polysaccharides and non-thiol-modified collagen components.

In other embodiments, the lipo-restoring composition is made by suspending the adipose derived cells in the resorbable matrix.

In other embodiments, the adipose derived cells are suspended by mixing.

In other embodiments, 5 ml of the lipo-restoring composition is administered to the face.

In other embodiments, between about 1 ml and about 5 ml of the lipo-restoring composition is administered to the face.

In other embodiments, between about 5 ml and about 10 ml of the lipo-restoring composition is administered to the face.

In other embodiments, between about 1 ml and about 20 ml of the lipo-restoring composition is administered to the face.

In other embodiments, between about 1 ml and about 40 ml of the lipo-restoring composition is administered to the face.

In other embodiments, between about 5 ml and about 15 ml of the lipo-restoring composition is administered to each side of the face.

In certain embodiments, the lipo-restoring composition comprises an implant.

In other embodiments, the adipose derived cells engraft after administration to a subject.

In certain embodiments, the engrafted cells vascularize.

In certain embodiments, the adipose derived cells progress to lipocytes after administration.

In certain embodiments, a method of one or more of slowing the progression of facial lipoatrophy, preventing facial volume decrease, restoring facial volume, increasing facial volume for greater than 6 months, or treating subcutaneous facial lipoatrophy defects in a subject includes administering a lipo-restoring composition to the face, where the lipo-restoring composition includes a combination of a resorbable matrix and fat.

In certain embodiments, the fat is from the subject or an unrelated subject.

In certain embodiments, a method of slowing the progression of facial lipoatrophy is described which includes administering a lipo-restoring composition to the face, wherein the lipo-restoring composition comprises a combination of a resorbable matrix and adipose derived cells.

In other embodiments, a method of preventing facial volume decrease is described which includes administering a lipo-restoring composition to the face, wherein the lipo-restoring composition comprises a combination of a resorbable matrix and adipose derived cells.

In yet other embodiments, a method of restoring facial volume is described which includes administering a lipo-restoring composition to the face, wherein the lipo-restoring composition comprises a combination of a resorbable matrix and adipose derived cells.

In further embodiments, a method of increasing facial volume for greater than 6 months is described and includes administering a lipo-restoring composition to the face, wherein the lipo-restoring composition comprises a combination of a resorbable matrix and adipose derived cells.

In another embodiment, a method of treating subcutaneous facial lipoatrophy defects is described which includes administering a lipo-restoring composition to the face, wherein the lipo-restoring composition comprises a combination of a resorbable matrix and adipose derived cells.

In another embodiment of the methods described herein, a female subject responds better than a male subject, wherein responding better comprises having a greater average facial volume increase at 6 months than a male.

In another embodiment of the methods described herein, a subject that does not consume alcohol responds better than a subject that does, wherein responding better comprises having a greater average facial volume increase at 6 months than a subject that does consume alcohol.

In another embodiment of the methods described herein, at 3 months after administration a subject has from about 54.2% to about 149.5% retention of volume as compared to one month after administration.

In another embodiment of the methods described herein, at 3 months after administration a subject has about 93.3% retention of volume as compared to one month after administration.

In another embodiment of the methods described herein, at 3 months after administration a subject has about 90.7% retention of volume as compared to one month after administration.

In another embodiment of the methods described herein, at 3 months after administration a subject has from about 83.1% to about 103.1% volume retained as compared to one month after administration.

In another embodiment of the methods described herein, at 6 months after administration a subject has from about 43.3% to about 115.6% retention of volume as compared to one month after administration.

In another embodiment of the methods described herein, at 6 months after administration about 85.7% of the volume administered was retained as compared to one month after administration.

In another embodiment of the methods described herein, at 6 months after administration about 89.2% volume was retained as compared to one month after administration.

In another embodiment of the methods described herein, at 6 months a subject has from about 43.3% to about 115.6% volume retained as compared to one month after administration.

In one embodiment, at 9 months, about 82% of the volume was retained.

In one embodiment, at 9 months, about 88% of the volume was retained.

In one embodiment, at 9 months about 76% to about 90%; from about 40% to about 92%; or from about 82% to about 88% volume was retained.

In one embodiment, at 12 months, about 70% of the volume was retained.

In one embodiment, at 12 months at least about 88% of the volume was retained.

In one embodiment, at 12 months from about 62% to about 84%; from about 30% to about 90%; or from about 70% to about 76% of the volume was retained.

In another embodiment of the methods described herein, at 12 months about 93% of the administered volume was retained as compared to one month after administration.

In another embodiment of the methods described herein, at 12 months about 100% of the administered volume was retained.

In another embodiment of the methods described herein, at 12 months after administration from about 84% to about 102% retention of volume was demonstrated in subjects.

In one embodiment, at 18 months, about 64% of the volume was retained.

In one embodiment, at 18 months at least about 68% of the volume was retained.

In one embodiment, at 18 months from about 56% to about 76%; from about 46% to about 78%; or from about 64% to about 68% of the volume was retained.

In one aspect, provided herein are methods of correcting moderate to severe facial wrinkles and folds, such as nasolabial folds or lip augmentation, comprising administering a lipo-restoring composition to the subcutaneous and/or supraperiosteal tissue of a subject.

In one aspect, provided herein are methods of augmentation to correct age-related volume deficit in the mid-face in comprising administering a lipo-restoring composition to the subcutaneous and/or supraperiosteal tissue of a subject.

In one embodiment, the subject is over the age of 21.

In one embodiment, the mid-face comprises the zygomaticomalar region, anteromedial cheek, and/or submalar region.

In one embodiment, the methods further comprise a touch-up treatment approximately 30 days after initial injection.

In one embodiment, the administering is by a multi-injection technique and/or in an antegrade or retrograde fashion.

In one embodiment, the multi-injection technique comprises tunneling, fanning, crosshatching, ferning, and serial puncture.

Further aspects of the technology described herein will be brought out in the following portions of the specification, wherein the detailed description is for the purpose of fully disclosing preferred embodiments of the technology without placing limitations thereon. Further aspects and embodiments are described infra.

### BRIEF DESCRIPTION OF THE DRAWINGS

The technology described herein will be more fully understood by reference to the following drawings which are for illustrative purposes only:

FIG. 1A through FIG. 1F show images of human stromal vascular fraction (SVF) cells differentiating into adipocyte clusters (FIG. 1F) in HyStem hydrogel.

FIG. 2 is a graph showing the mean hemifacial incremental volume evolution over 6 months for the randomized subjects.

FIG. 3 shows a graph of the mean hemifacial incremental volume evolution over 12 months for the subjects.

FIG. 4 shows an image of a histological section of a biopsy taken from a subject stained with H/E+Oil Red O and showing the formation of adipose cells.

FIG. 5 shows an image of a histological section of a biopsy taken from a subject stained with CD31+ and showing vascularization.

FIG. 6 shows the differences in hemifacial volume retention for the subgroups: males versus females and those subjects who drink alcohol versus those subjects that do not drink alcohol for both control and procedure subjects.

FIG. 7 shows the mean incremental hemifacial volume for baseline and follow-up months 1, 3, 6, 9, 12 and 18 for the treatment group.

FIG. 8A shows an example of a hyaluronan and gelatin (e.g., Glycosil-Gelin) component of a resorbable matrix and a linking agent (e.g., Extralink).

FIG. 8B shows an illustration of fat tissue being harvested by liposuction.

FIG. 8C shows thawed Hyaluronan and gelatin combined with Extralink to form the Renevia hydrogel in a syringe, according to certain embodiments.

FIG. 8D shows what the Hyaluronan and gelatin component looks like when Extralink is added and allowed to gel, according to some embodiments.

FIG. 9A shows the pre-gelled Renevia hydrogel, according to certain embodiments.

FIG. 9B shows the Renevia hydrogel after gelation, according to certain embodiments.

FIG. 10 shows an example of a resorbable matrix (Renevia) mixed with fat. Yellow fat parcels can be seen evenly distributed throughout the gel.

FIG. 11 shows an image of fat alone (bottom syringe) and hydrogel+fat (top syringe) when extruded from a syringe. As shown, the hydrogel-fat mixture gels after extrusion.

FIG. 12 a diagram of an HA based resorbable matrix cross-linking in situ.

FIG. 13A shows a diagram of the Coleman technique.

FIG. 13B shows bolus injections in a pig ear.

FIG. 14 shows a diagram of biopsy positions on each implant in a pig model.

FIG. 15 shows a diagram showing Coleman vs. bolus implantation positions.

### DETAILED DESCRIPTION

Described herein, in some embodiments are compositions and methods of treating lipoatrophies.

Before explaining at least one embodiment in detail, it is to be understood that the embodiments are not necessarily limited in its application to the details set forth in the following description or exemplified by the Examples. The methods and devices described herein are capable of other embodiments or of being practiced or carried out in various ways.

Current treatment options for lipoatrophy are limited, expensive and short term. Treatment options range from recombinant growth hormones to surgery using various implants both synthetic and developed from human tissue. Although these currently available implants range from temporary to semi-permanent, the results are highly variable.

The present disclosure provides compositions and methods for correcting soft tissue defects caused by lipoatrophy in a subject by restoring tissue to the lipoatrophic area. In certain embodiments, the disclosure provides methods for restoring and regenerating soft tissue in a subject using resorbable matrices to deliver cells to the affected area. For cell-based tissue engineering applications, it is beneficial to create a 3-dimensional space in which the implanted cells can attach, proliferate, and differentiate. While it has been clearly shown in animal studies that adipose derived stromal vascular cells can differentiate into a wide variety of cell types including adipocytes, a functional resorbable delivery matrix provides an advantage by creating a temporary space for tissue formation. In certain embodiments, the addition of fat can be used for larger volume correction of facial volume deficit.

In certain embodiments, hydrogels that have all of the characteristics required for successful delivery of complex, fragile cells and macromolecules can be used as the resorbable matrix.

Recently, a family of hyaluronan based hydrogels (trade named HYSTEM® and RENEVIA®) have been developed that mimic the natural extracellular matrix environment (ECM) for applications in 3-D cell culture, stem cell propagation and differentiation, tissue engineering, regenerative medicine, and cell based therapies. HyStem and Renevia hydrogels were designed to recapitulate the minimal composition necessary to obtain a functional extracellular matrix. The individual components of the hydrogels are cross-linkable in situ, and may be seeded with cells prior to injection in vivo, without compromising either the cells or the recipient tissues.

In various embodiments, the hydrogels contemplated herein are designed to crosslink into the hydrogel form, for example, starting from a liquid form after it is injected into the body. In some embodiments, the hydrogel begins to crosslink and is becoming more viscous as it is being administered. Other hydrogels or tissue engineering approaches use semi-rigid substances that must be implanted. The liquid and delayed self-assembly of these hydrogels and the surprising discovery that they permit injection without shearing forces that would destroy cells allows a very small needle to be used for delivery of cells into the body. In some embodiments, a 30 gauge syringe needle may be used.

The technology underlying HyStem hydrogels is based on a unique thiol cross-linking strategy to prepare hyaluronan based hydrogels from thiol-modified hyaluronan and other ECM constituents. Building upon this platform, a family of unique, biocompatible resorbable hydrogels have been developed. The building blocks for HyStem and Renevia hydrogels are hyaluronan and gelatin, each of which has been thiol-modified by carbodiimide mediated hydrazide chemistry. Hydrogels are formed by cross-linking mixtures of these thiolated macromolecules with polyethylene glycol diacrylate (PEGDA) (see U.S. Pat. Nos. 7,928,069 and 7,981,871, incorporated herein by reference in their entirety). The rate of gelation and hydrogel stiffness can be controlled by varying the amount of cross-linker. An attribute of these hydrogels is their large water content, >98%, resulting in high permeabilities for oxygen, nutrients, and other water-soluble metabolites.

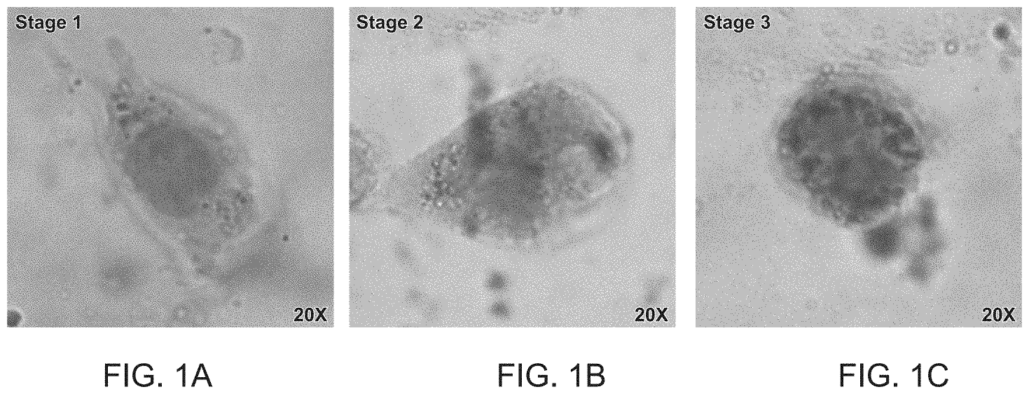

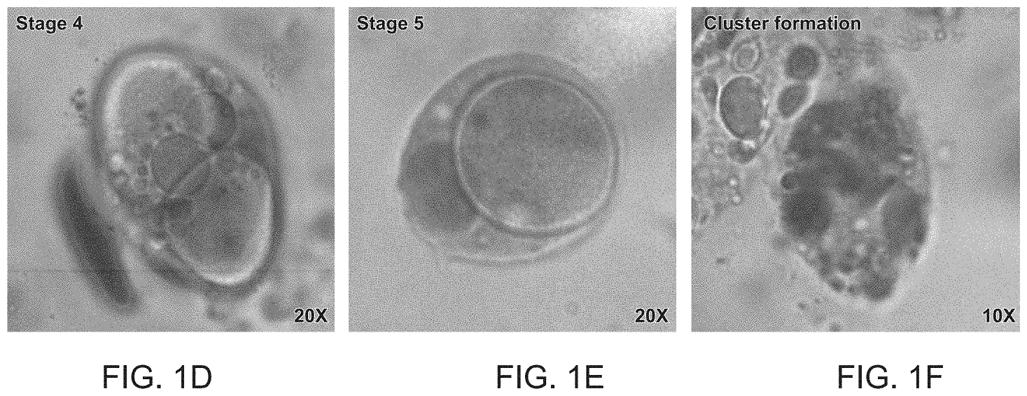

Hydrogels, such as HyStem and Renevia, have been shown to support attachment and proliferation of a wide variety of cell types in both 2-D and 3-D cultures and exhibit a high degree of biocompatibility in animal studies when implanted in vivo. These hydrogels are readily degraded in vitro and resorbed in vivo through hydrolysis via collagenase and hyaluronidase enzymes. When implanted in these hydrogels, cells remain attached and localized within the hydrogel and slowly degrade the implanted matrix replacing it with their natural ECMs. FIG. 1A through FIG. 1F shows human stromal vascular fraction (SVF) cells differentiating into adipocyte clusters (FIG. 1F) in HyStem hydrogel.

Hydrogels, such as HyStem and Renevia, can offer an advantageous replacement for autologous fat as the scaffold for autologous cell assisted lipotransfer (CAL) transplant procedures. The hyaluronate component of the hydrogel provides the necessary 3-dimensional space filling framework while the gelatin component provides the requisite amino acid sites for cell attachment and proliferation. These properties, coupled with the ability to uniformly mix the stromal vascular cells with hydrogel components and deliver the cell/scaffold mixture through a small gauge needle (21-30 gauge) for in situ gelation, can greatly reduce complications associated with adipose tissue processing, survival and delivery using CAL. As an implantable scaffold, the resorbable matrices described herein can provide a safe and consistently uniform matrix with which to deliver minimally manipulated, autologous stromal vascular cells for tissue augmentation procedures and for the treatment of contour defects. Additionally, the compliance (stiffness) of hydrogels can be formulated to ˜70+20 Pa which is similar to adipose tissue.

In some embodiments, the biocompatible resorbable matrix composition can have a storage modulus of about 1 Pa to about 5 Pa, about 1 Pa to about 5,000 Pa, about 20 Pa to about 5,000 Pa, about 50 Pa to about 5,000 Pa, about 60 Pa to about 1,200 Pa, about 75 Pa to about 1,000 Pa, about 80 Pa to about 120 Pa, about 15 Pa to about 100 Pa, about 20 Pa to about 150 Pa, or any value in a range bounded by, or between, any of these values.

In some embodiments, the lipo-restoring composition is administered while the lipo-restoring composition has a storage modulus of between about 0.1 Pa and about 10 Pa and wherein the lipo-restoring composition continues to cure in situ to between about 20 Pa to about 150 Pa.

In some embodiments, the hydrogel may contain cellular attachment sites to prevent anoikis of anchorage-dependent cells. They may also have functionalizable groups on its component biopolymers allowing not only the one-step covalent linking of macromolecular therapeutic cargo by the user, but also provide for matrix customization for specific cell types requiring a unique collection of cellular attachment sites. Finally, the hydrogels described infra may have validated and desired syringeability with the gauge of the needle determined by the placement location. These properties may be achieved by varying the concentration of one or more of the monomers and/or the oxidizing agent.

Where the therapeutic agent is a cell, the cell may attach to a gelatin component of a hydrogel. Alternatively, the cell may be attached to a functionalized monomer within the hydrogel, such as peptide functionalized monomer. Suitable peptides may comprise the RGD sequence.

In certain embodiments, the biocompatible resorbable matrix composition may be mixed with adipose derived cells to be administered to a subject in need of soft tissue regeneration. In some embodiments, the biocompatible resorbable matrix/cell composition may be administered about 5 minutes to about 180 minutes, about 10 minutes to about 150 minutes, or about 20 minutes to about 120 minutes post mixing of the components and prior to the final crosslinking or curing of the biocompatible resorbable matrix/cell composition. In some embodiments, the biocompatible resorbable matrix/cell composition has a storage modulus of between about 1 Pa and about 10 Pa at the time the biocompatible resorbable matrix/cell composition is administered to the subject and a storage modulus of about 50 Pa to about 150 Pa once the biocompatible resorbable matrix/cell composition crosslinks or cures, in situ.

In various embodiments, the lipo-restoring composition is administered when the lipo-restoring composition is at about G′ 1 to about 5 Pa; or at about 0.3 to about 20 Pa; or at about 0.5 to about 10 Pa; or at about 0.75 to about 7.5 Pa. The lipo-restoring composition is administered when the lipo-restoring composition is at about 1 to about 5% of its final stiffness; or about 0.1 to about 50% of its final stiffness; about 5 to about 75% of its final stiffness; or about 2 to about 4% of its final stiffness.

In certain embodiments, the resorbable matrix crosslinks before, during and/or after administration. In others, the resorbable matrix crosslinks before, during and/or after the SVF is mixed with the resorbable matrix.

In certain embodiments, the resorbable matrix begins to crosslink before the SVF is mixed with the resorbable matrix.

In certain embodiments, the resorbable matrix continues to crosslink after administration of the lipo-restoring composition.

In certain embodiments, the lipo-restoring composition is administered by injection.

In certain embodiments, the lipo-restoring composition is administered about 5 to about 40 minutes, about 10 to about 30 minutes or 15 to about 20 minutes post mixing of components.

In certain embodiments, the components comprise, SVF, a thiol-modified hyaluronan and a thiol-modified collagen.

In certain embodiments, the components further comprise a crosslinker.

In certain embodiments, the crosslinker comprises one or more of bi-, tri-, multi-functionalized molecules that are reactive to thiols, and/or oxidation agents that initiate crosslinking.

In certain embodiments, the crosslinker comprises polyethylene glycol diacrylate.

In certain embodiments, the thiol-modified hyaluronan has a molecular mass of at least 55000 g/mol; at least 100,000 g/mol; at least 120,000 g/mol; at least 150,000 g/mol; at least 170,000 g/mol; at least 175,000 g/mol; or at least 200,000 g/mol.

In certain embodiments, the thiol-modified hyaluronan comprises more than 150 μmol/g of polymer; more than 200 μmol/g of polymer; more than 1000 μmol/g of polymer; more than 10,000 μmol/g of polymer.

In certain embodiments, the thiol-modified hyaluronan comprises from about 1% to about 75% of the thiol groups in the resorbable matrix.

In certain embodiments, the thiol-modified collagen comprises from about 1% to about 75% of the thiol groups in the resorbable matrix.

In one embodiment, the resorbable matrix crosslinks before, during and/or after administration.

In one embodiment, the resorbable matrix crosslinks before, during and/or after the SVF is mixed with the resorbable matrix.

In one embodiment, the resorbable matrix begins to crosslink before the SVF is mixed with the resorbable matrix.

In one embodiment, the resorbable matrix continues to crosslink after administration of the lipo-restoring composition.

In one embodiment, the lipo-restoring composition is administered by injection.

In one embodiment, the lipo-restoring composition is administered about 5 to about 40 minutes, about 10 to about 30 minutes or 15 to about 20 minutes post mixing of components.

In one embodiment, the components comprise, SVF, a thiol-modified hyaluronan and a thiol-modified collagen.

In one embodiment, the components further comprise a crosslinker.

In one embodiment, crosslinker comprise one or more of bi-, tri-, multi-functionalized molecules that are reactive to thiols, and/or oxidation agents that initiate crosslinking.

In one embodiment, the crosslinker comprises polyethylene glycol diacrylate.

In one embodiment, the thiol-modified hyaluronan has a molecular mass of at least 55000 g/mol; at least 100,000 g/mol; at least 120,000 g/mol; at least 150,000 g/mol; at least 170,000 g/mol; at least 175,000 g/mol; or at least 200,000 g/mol.

In one embodiment, the thiol-modified hyaluronan comprises more than 150 μmol/g of polymer; more than 200 μmol/g of polymer; more than 1000 μmol/g of polymer; more than 10,000 μmol/g of polymer. In some embodiments the SLF has a thiol content of from about 24-96 μmoles thiol/vial.

In one embodiment, the thiol-modified collagen comprises from about 1% to about 75% of the thiol groups in the resorbable matrix.

In one embodiment, the thiol-modified hyaluronan comprises from about 1% to about 75% of the thiol groups in the resorbable matrix.

In certain embodiments, the thiolation levels of the thiol-modified hyaluronan component of a resorbable matrix are in the range of between about 0.01 to about 1.0 μmoles/mg. In other embodiments, the thiolation levels of the thiol-modified gelatin component of a resorbable matrix are in the range of between about 0.01 to about 3.0 μmoles/mg. In still other embodiments wherein the resorbable matrix comprises a hyaluronan component and a gelatin component, the thiolation levels of the thiol-modified hyaluronan/gelatin composition comprises between about 0.001 to about 3.0 μmoles/mg.

In one embodiment, the lipo-restoring composition is administered when the lipo-restoring composition is at about G′ 1 to about 5 Pa; or at about 0.3 to about 20 Pa; or at about 0.5 to about 10 Pa; or at about 0.75 to about 7.5 Pa.

In one embodiment, the lipo-restoring composition is administered when the lipo-restoring composition is at about 1 to about 5% of its final stiffness; or about 0.1 to about 50% of its final stiffness; about 5 to about 75% of its final stiffness; or about 2 to about 4% of its final stiffness.

Crosslinkers may comprise, for example, a bi-, tri-, multi-functionalized molecule that is reactive to thiols (e.g. maleimido groups), oxidation agents that initiate crosslinking (e.g., GSSG), glutaraldehydes, and environment influences (e.g., heat, gamma/e-beam radiation). In some embodiments, there are no cross-linkers necessary. In some embodiments, the crosslinking agent is not present in the final hydrogel composition.

Renevia is an example of an implantable resorbable matrix that can be used in certain embodiments described in the present disclosure. Renevia can be in a lyophilized format comprised of four components—individual vials of thiol-modified hyaluronan, thiol-modified gelatin, crosslinker (e.g., polyethylene glycol diacrylate), and a user-supplied vial of sterile water for reconstitution. In this example, there are four separate components that must be combined. The lyophilized components (hyaluronan and gelatin) are heated at 37° C. shaking incubator to reconstitute the components. Each vial also requires from 2 minutes to about 5 minutes or, from 2 minutes to about 60 minutes or from 30 to 60 minutes to reconstitute. Lyophilized product can be supplied in 5 cc, 10 cc, 15 cc, 20 cc, 25 cc, 30 cc, 35 cc and 50 cc or from between 5 cc to about 50 cc final reconstituted volume amounts.

In some embodiments, the resorbable matrix may be reconstituted by transferring about 2 cc of a reconstitution buffer into a vial containing a thiol-modified gelatin, transferring about 2 cc of a reconstitution buffer into a vial containing a thiol-modified hyaluronan, transferring about 1 cc of a reconstitution buffer into a vial containing a crosslinking agent, incubating the vials at a temperature of about 37° C. and shaking the vials at about 150 RPM for at least about 30 minutes.

In one embodiment, the resorbable matrix is lyophilized and for reconstitution from three vials. After reconstitution, hyaluronic acid will be from 0.1 to about 5% of the resorbable matrix. Gelatin will be from 0.1 to about 5% of the resorbable matrix. Other components of the resorbable matrix will be one or more salts (e.g., sodium and potassium salts) that will be from 0.01 to about 5% and phosphates (e.g., sodium and potassium phosphates) that will be from 0.01 to about 2%. transfer 2 cc of sterile water for injection into the Gelin vial.

In certain embodiments, the resorbable matrix comprises a kit comprising, 1 vial hyaluronan and gelatin vial (10.7 mL Frozen Liquid; in phosphate buffered saline (PBS), pH 7.4 (Frozen), Hyaluronic acid, thiolated (4 mg/mL), Porcine gelatin, thiolated (4 mg/mL), Sodium chloride (8 mg/mL), Potassium chloride (0.2 mg/mL), Sodium phosphate, dibasic, heptahydrate (2.72 mg/mL), Potassium phosphate, monobasic (0.24 mg/mL)), 1 vial Extralink Vial (20 mg bis-acrylated polyethylene glycol Lyophilized Solid).

In certain embodiments, the biocompatible resorbable matrix comprises a polysaccharide based polymer, (for example, a hyaluronan based, chitosin based) with a polysaccharide concentration of about 1 mg/mL to about 20 mg/mL, about 2 mg/mL to about 10 mg/mL, about 3 mg/mL, about 4 mg/mL, or about 5 mg/mL.

In certain embodiments wherein the resorbable matrix includes a hyaluronic acid component, the hyaluronic acid has a molecular weight of between about 10,000 to about 10,000,000 Da, about 25,000 to about 5,000,000 Da, about 50,000 to about 3,000,000 Da. In another embodiment, the hyaluronic acid has a molecular weight in the range of between about 300,000 and about 3,000,000 Da, about 400,000 and about 2,500,000 Da, about 500,000 and about 2,000,000 Da, about 600,000 and 1,800,000 Da. In other embodiments, the hyaluronic acid has a molecular weight of between about 10,000 and about 800,000 Da, about 20,000 and about 600,000 Da, about 30,000 and about 500,000 Da, about 40,000 and about 400,000 Da, about 50,000 and about 300,000 Da.

In other embodiments, the hyaluronic acid component can comprise an inorganic salt of hyaluronic acid, including but not limited to, sodium hyaluronate, potassium hyaluronate, ammonium hyaluronate, calcium hyaluronate, magnesium hyaluronate, zinc hyaluronate, or cobalt hyaluronate.

In other embodiments, the biocompatible resorbable matrix includes a gelatin component (for example, collagen) with a gelatin concentration of between about 1 mg/mL to about 20 mg/mL, about 2 mg/mL to about 10 mg/mL, about 3 mg/mL, about 4 mg/mL, or about 5 mg/mL.

In certain embodiments where the biocompatible resorbable matrix comprises a hyaluronan and gelatin hydrogel composition, the hyaluronan:gelatin weight ratio can be between 1:1 and 10:1; the hyaluronan:gelatin weight ratio can be between about 1:1 to about 1:10; about 1:1.5; about 1.5:1; about 1:2; about 2:1; or from between about 0.5:5 to about 5:0.5.

In certain embodiments wherein the resorbable matrix comprises a hyaluronan and gelatin hydrogel composition with a crosslinking agent, the crosslinking agent may comprise a weight ratio of between about 1:1, and 100:1 of hyaluronan/gelatin:crosslinking agent, and preferably about 2:1 to about 50:1 of hyaluronan/gelatin:crosslinking agent (dry weight).

Certain procedures could be simplified if only one kit per procedure was required instead of two. In one embodiment, the hyaluronan component and the collagen, or gelatin components are supplied as a liquid mixture in one vial. The liquid mixture may be frozen in one embodiment. First, this stable liquid format (SLF) reduces the number of components from 4 to 2 since one vial now contains a hyaluronan component/collagen, or gelatin components mixture and sterile reconstitution solution is no longer required. Second, since refrigerators and freezers are typical equipment in a medical setting, no new equipment is needed for purchase and set-up. While some time is required to thaw the frozen liquid, this step is less cumbersome since the SLF either can be thawed at the time of use at room temperature or in a water bath; alternatively, it can be thawed at refrigerated temperatures. Third, SLF kits can provide 10 cc of material.

The collagen, in some embodiments comprises a porcine derived collagen. In other embodiments, the collagen comprises human, bovine, porcine, or other mammalian derived collagen. In some embodiments, different collagens may be used. For example, collagen type I, collagen type III, collagen type IV, collagen type VI, or a combination thereof, may be used. Some embodiments comprise collagen that has been denatured to gelatin.

An example of a SLF comprises 80 mg (in, for example, 10 ml) of hyaluronan/gelatin mixture, wherein there are 40 mg of hyaluronan and 40 mg of gelatin. In another example of SLF, there are from 0.025 to 200 mg of hyaluronan and 0.025 to 200 mg of gelatin in a vial.

TABLE 1
SLF Formulations

| Description | Quantity | | Quantity | Quantity | | Quantity | |
|---|---|---|---|---|---|---|---|
| thiol-modified | 40 | mg | 0.01 mg-3000 g | 0.01 | | 5-1000 | mg |
| hyaluronan | | | | | | | |
| thiol-modified | 40 | mg | 0.01 mg-10000 g | 80 | mg | 5-1000 | mg |
| collagen | | | | | | | |
| water | QS | | QS | QS | | QS | |
| Sodium | 80 | mg | 0-20000 g | — | | 0-1000 | mg |
| Chloride | | | | | | | |
| Potassium | 2 | mg | 0-20000 g | — | | 0-100 | mg |
| Chloride | | | | | | | |
| Sodium | 27.2 | mg | 0-20000 g | — | | 0-500 | mg |
| Phosphate | | | | | | | |
| Dibasic | | | | | | | |
| Potassium | 2.4 | mg | 0-20000 g | — | | 0-500 | mg |
| Phosphate | | | | | | | |
| Monobasic | | | | | | | |
| Salt | — | | 0-20000 g | — | | 0-20000 | g |
| Volume | 10 | ml | 0.25 ml-10 L | 10 | ml | 100 | ml |

In other embodiments, the resorbable matrix includes an SLF composition with non-thiol-modified polysaccharides. In other embodiments, the resorbable matrix includes an SLF composition with non-thiol-modified collagen or gelatin, for example. In yet other embodiments, the resorbable matrix includes an SLF composition with both non-thiol-modified polysaccharides and non-thiol-modified collagen components.

Although specific examples of hydrogels that are suitable for providing resorbable matrices are described for use with embodiments of the present disclosure, it will be understood that any suitable resorbable cellular matrix may be used. For example, gels made using oxidized glutathione (GSSG) as a cross-linking agent may be used (see US Patent Application Publication No. US 2014-0341842, incorporated herein by reference in its entirety).

The SLF may comprise a pH of from 7 to about 8. In some embodiments, the pH is between about 7.2 and about 7.6.

In some embodiments, the SLF resorbable matrix composition may be stored from between −80 degrees C. to about 45 degrees C., or from between −20 degrees C. to about 25 degrees C., from between −10 degrees C. to about 4 degrees C., or from between 0 degrees C. to about 10 degrees C.

Methods of the present disclosure can be carried out, for example, by harvesting a subject's adipose derived cells, combining the harvested cells with a biocompatible resorbable matrix and implanting the cell/matrix composition into an area of the subject that has been affected by lipoatrophy. The cell/matrix mixture can provide a lipo-restoring composition.

In certain embodiments, the adipose derived cells that can be used for implantation include, but are not limited to, autologous adipose derived cell, stromal vascular cells, stromal vascular fraction, multipotent stem cells, pre-adipocytes, and endothelial precursor cells. In some embodiments, the adipose derived cells can be derived from the subject's own or another subject's stem cells. The cells can be derived according to methods known in the art. In some embodiments, the cells can be genetically modified using methods known in the art.

In some embodiments, adipose tissue, fat tissue, or “fat” may include loose fibrous connective tissue comprising fat cells (adipocytes) and multiple types of regenerative cells, and may comprise brown and/or white adipose tissue. It may be harvested from any body site, such as, for example, subcutaneous, omental/visceral, interscapular, or mediastinal. It may be obtained from any subject having adipose tissue.

In some embodiments, the stromal vascular fraction (SVF) to resorbable matrix volume ratio can be about 1:1, about 1:2, about 1:3, about 1:4, about 1:5, about 1:6, about 1:7, about 1:8, about 1:9, or about 1:10. In some embodiments, about 4×10⁷ to about 9×10⁷ SVF cells may combined with the resorbable matrix and injected into the subject. In other embodiments, between about 6×10⁷ and about 8×10⁷ viable SVF cells may be combined with the resorbable matrix.

In other embodiments, fat can be combined with the resorbable matrix, which results in enhanced handling and sculptability over fat alone. In addition, longer lasting volume effects can be seen and lower volumes of lipoaspirates are required, which enables the procedure to be performed as an in-office procedure. In certain embodiments, the fat:resorbable matrix weight to weight ratio (wt fat/wt matrix) can be between about 0.1 to about 5. All fat to resorbable matrix weight ratios falling within this range are considered to be within the scope of the present disclosure.

In some embodiments, the fat or fat tissue to resorbable matrix volume ratio can be about 1:1, about 2:1, about 3:1, about 4:1, about 5:1, about 6:1, about 7:1, about 8:1, about 9:1, or about 10:1. In some embodiments, about 4×10⁷ to about 9×10⁷ fat cells may combined with the resorbable matrix and injected into the subject. In other embodiments, between about 6×10⁷ and about 8×10⁷ viable fat cells may be combined with the resorbable matrix.

In certain embodiments, the hyaluronan and the gelatin and/or collagen component of the resorbable matrix is thawed and combined aseptically with a linking agent (e.g., Extralink) to form the resorbable matrix, at the point of administration. In another embodiment, the resorbable matrix is reconstituted from lyophilized vials of components. The resorbable matrix may be injected into the area of volume deficit per instructions provided and/or by methods known in the art of medical practice. In other embodiments, the health care provider can extract a small volume of autologous tissue preparation (e.g., lipoaspirate) from the subject per instructions provided and/or according to techniques know to those of skill in the art. The tissue preparation may be mixed with resorbable matrix prior to full gelation, during gelation, or prior to gelation and then injected in the target area of volume deficit.

Target areas of volume deficit, include the face, including, for example, the subcutaneous and/or supraperiosteal tissue of a subject. In some embodiments, the volume deficit is in the mid-face, which comprises mid-face the zygomaticomalar region, anteromedial cheek, and/or submalar region.

In certain embodiments, the administration is by multi-injection technique and/or in an antegrade or retrograde fashion, which may include, for example, tunneling, fanning, crosshatching, ferning, and serial puncture.

In certain embodiments, the resorbable matrix crosslinking process is relatively insensitive to the pH of tissues that are present in non-buffered solutions. This is because the resorbable matrix formulation can be buffered in 1× PBS (pH 7.4) which may be sufficient to maintain the pH in the presence of added lipoaspirate fat present in unbuffered solutions (e.g., 0.9% NaCl this may be used during the liposuction). In addition, the gelation time may be increased by several minutes (e.g., from 20 to 30 minutes or 90 minutes) when the gel is mixed with tissue in 3:1 volumetric ratio, for example wherein the gel:tissue preparation of 3:1 or greater.

Fat is easily entangled within crosslinked resorbable matrices made of HA, for example. This is because the diameter of the fat parcel in lipoaspirated fat is at least approximately 2 mm, which is the smallest diameter of the distal end of a 10 mL syringe through which the fat may pass prior to gelation with the resorbable matrix. Since the pore size of HA for example, according to scanning electron microscopy, is less than 400 microns (0.4 mm), an HA based hydrogel resorbable matrix can retain fat parcels and keeps them in place.

In one embodiment, the components of the resorbable matrix are frozen, and can be removed from the freezer and thawed for about 2 minutes to about 90 minutes prior to treatment. The hyaluronic acid and collagen component flows like water when thawed. While the components are thawing, the clinician can obtain fat from the patient via a procedure of fat removal known in the art. The autologous fat particles can be processed to preserve the function and characteristics of whole adipose tissue, washed or otherwise processed as known in the art. In one embodiment, the fat tissue that is added to the resorbable matrix is only mechanically disrupted and remains incorporated in an extracellular matrix.

As an example of a preparation of a particular resorbable matrix appropriate for use with fat, hyaluronic acid and collagen may be combined aseptically with a crosslinker to form the a resorbable hydrogel. The reaction may proceed without byproducts or changes in temperature or pH, and can occur in situ. During the cross-linking process, the molecules interact with each other to create the three-dimensional hydrogel matrix. After combination of the resorbable hydrogel components, the combination may be mixed with fat (prior to gelation or during gelation), and drawn into a syringe and an appropriately gauged needle. The hydrogel-fat mixture is allowed to cure for between about 2 minutes and about 120 minutes after the resorbable hydrogel components have been combined. Homogeneity can be enhanced by regular inversion or rotation of the syringe. Gel consistency can be checked for signs of gelation. In this step, the fat tissue particles become physically encapsulated/entangled within the forming hydrogel. Once gelation starts, the hydrogel-fat mixture can be injected within about 2 to about 120 minutes via surgical procedures known to one of skill in the art (e.g., various techniques for injecting dermal fillers or fat may be employed).

In one embodiment, 5 ml of cell/resorbable matrix may be administered to the subject. However, this volume can be adjusted according to the condition to be treated. For example, volumes of about 1 ml to about 40 ml may be administered. In addition, the cell/resorbable matrix can be administered to each side of the face in varying volumes. Furthermore, the cell/resorbable matrix can be administered to other areas of the body in need thereof.

In another embodiment, fat/resorbable matrix compositions can also be administered to a subject at a volume of 5 ml. However, this volume can be adjusted according to the condition to be treated. For example, volumes of about 1 ml to about 40 ml may be administered. In addition, the fat/resorbable matrix can be administered to each side of the face in varying volumes. Furthermore, the fat/resorbable matrix can be administered to other areas of the body in need thereof.

Examples of lypoatrophies that can be treated according to embodiments described herein include, but are not limited to, facial lipoatrophies, mid-face volume deficit, mild to severe submalar volume deficit, mild to severe perioral volume deficit, and/or subcutaneous contour defects. These defects can arise from, for example, HIV infection or HAART treatment, secondary congenital abnormalities, trauma, surgical resection, the aging processes, and diseases such as diabetes, auto immune disease, acquired generalized lipodystrophy (AGL), Lawrence syndrome, acquired partial lipodystrophy (APL), progressive lipodystrophy, Barraquer-Simons syndrome, injury, weight loss, repeated injection site, localized pressure or infections caused by one or more of measles, pneumonia, infectious mononucleosis, or hepatitis.

Because the compositions of cells and resorbable matrices described herein elicit the attachment, proliferation and differentiation of administered cells, treatment results can be long lasting, such as greater than 18 months.

The effectiveness of treatment may be assessed by different measures of structure, function and aesthetics, including, among others, effectiveness of volume restoration as gauged by the scoring of the MFVDS by independent evaluators and GAIS by subject and independent evaluators using high quality digital photography, subject-reported outcomes including the BIQLI-SP and the Rosenberg Self Esteem Score, skin type using the Fitzpatrick Skin Type Scale, Pain as indicated by the VAS pain Score, Skin thickness as measured by ultrasound, and 3D Image Scan. Assessment can be made before, during or after treatment.

The resorbable matrix may comprise lyophilized powders or frozen liquids or a combination. The contents of each vial or other container may be, for example, sterile and nonpyrogenic. Resorbable matrix kits may be stored frozen or at room temperature, for example between −25° C. to −10° C. or between 4° C. to 25° C. until ready for use.

In certain embodiments, the resorbable matrix comprises a kit comprising, 1 vial Hyaluronan and gelatin Vial (10.7 mL Frozen Liquid; in phosphate buffered saline (PBS), pH 7.4 (Frozen), Hyaluronic acid, thiolated (4 mg/mL), Porcine gelatin, thiolated (4 mg/mL), Sodium chloride (8 mg/mL), Potassium chloride (0.2 mg/mL), Sodium phosphate, dibasic, heptahydrate (2.72 mg/mL), Potassium phosphate, monobasic (0.24 mg/mL)), 1 vial Extralink Vial (20 mg bis-acrylated polyethylene glycol Lyophilized Solid).

In other embodiments, where the resorbable matrix comprises a hyaluronan, gelatin and/or a crosslinking component, the components may be provided in separate vials comprising volumes of about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, and/or about 25 mL. In certain embodiments, the components are in a lyophilized or powder form, which can be reconstituted prior to use or combination with additional components.

For reconstitution, if necessary, the resorbable matrix components may be removed from storage and opened. Using, for example, a syringe, to transfer water into the vial.

In other embodiments, the resorbable matrix comprises a kit comprising one or more vials of a lyophilized gelatin (e.g., collagen) component at between about 5 mg to about 100 mg, a lyophilized hyaluronic acid component at between about 5 mg to about 100 mg, a lyophilized crosslinking agent at between about 1 mg to about 50 mg and a reconstitution solution at between about 1 mL to about 30 mL.

In other embodiments, the lyophilized resorbable matrix includes a composition with non-thiol-modified polysaccharides. In other embodiments, the resorbable matrix includes a composition with non-thiol-modified collagen or gelatin, for example. In yet other embodiments, the resorbable matrix includes a composition with both non-thiol-modified polysaccharides and non-thiol-modified collagen components.

Harvested lipoaspirate is processed to obtain approximately 100-200 cc of dry adipose tissue and a cellular suspension is obtained, free of tissue fragments or fibers. In another embodiment, between about 25 to about 400 cc of dry adipose tissue is obtained, or any value in between. In another embodiment, between about 75 to about 250 cc of dry adipose tissue is obtained.

The SVF cell pellet may be resuspended, for example using a syringe (2.5 cc, 5 cc, 10 cc, 15 cc, 20 cc, 50 cc, 100 cc or other size syringe). The needle may be, for example, a 14 g spinal needle or a 15, 16, 18 or other gage needle. The cell pellet may be resuspended, for example in from about 2 cc to about 50 cc of solution, or about 10 cc of solution, for example, lactated ringer's.

The SVF cell suspension may be divided into, for example, 15 mL tubes, or other suitable containers may be used. The resuspended cells are centrifuged for from about 1 minute to about 10 minutes at from about 100 to about 700×g.

As much liquid as possible is removed without disturbing the SVF cell pellet. Using a syringe with an 15-25 g needle, the resorbable matrix is prepared and the SVF cell pellet is re-suspended to form the lipo-restoring composition. The lipo-restoring composition may be swirled by hand or mechanically until the SVF cells are well suspended, or if needed, repeatedly drawn into a syringe fitted with a, for example, 14 g spinal needle (for example, 3-5 times or 1 to 15 times, or any value in-between).

The cell/resorbable matrix mix (e.g., the lipo-restoring composition) is drawn from one centrifuge tube into a syringe fitted with a needle, for example, a 14 g spinal needle of 5.5-6 inches. The lipo-restoring composition may be distributed into smaller syringes, if desired. Syringes may be placed in an incubator with or without agitation. The syringe may be maintained at a temperature of about 37° C. The syringe may also be in motion, constant motion or still. For example, constant motion (100-150 rpm) may be about 10 minutes to allow cell/resorbable matrix mix to begin gelation before injection. The syringes can be left in the sterile tray that previously contained the GID device.

The cell/resorbable matrix mix may be injected within about 1 minute to about 12 hours of mixing, or from about 1 minutes to about 60 minutes of mixing. When the gel is becoming viscous (viscosity may be checked, for example, every 1 to about every 10 minutes to determine how the consistency of the gel is changing before beginning the injection).

The skin area may be washed and disinfected with, for example, alcohol or other antiseptic. Prior to the injection, the needle may be changed with a 21 to 32 g needle, for example. The needle may be from 14 to about 32 gauge, or from 15 to about 32 gauge. Various injection techniques may be employed that vary needle angle, bevel orientation, injection depth, and injection volume. A linear threading method, for example, and/or serial punctures may be utilized. The injection area may be massaged if needed to increase conformity with the injection site contours.

### EXAMPLES

The following examples are not intended to limit the scope of what the inventors regard as their invention nor are they intended to represent that the experiments below are all or the only experiments performed.

### Example 1

### A Randomized, Evaluator-Blinded, Delayed-Treatment-Controlled Study of the Effectiveness and Safety of Renevia as a Resorbable Matrix for the Delivery of Autologous Adipose Derived Cells to Treat Subcutaneous Facial Lipoatrophy Defects

The purpose of the study was to determine the effectiveness and safety of Renevia as a resorbable matrix for the delivery of autologous adipose derived cells (also known as stromal vascular fraction, or SVF) to treat subcutaneous facial lipoatrophy associated with anti-retroviral therapy for HIV infection. The SVF cells are composed of multipotent stem cells and precursors of fat cells (pre-adipocytes), all of which are important for the formation of new adipose tissue (Zuk, P. A., et al., Multilineage cells from human adipose tissue: implications for cell-based therapies. Tissue Eng, 2001. 7(2): p. 211-28.). Renevia can serve as a temporary three-dimensional matrix in which the implanted cells (which can be obtained from the patients themselves) can attach, proliferate, and differentiate into fully-formed fat cells (adipocytes). Over time this matrix is absorbed by the body and replaced with natural extracellular matrix.

FIG. 1A through FIG. 1F shows differentiation of SVF into adipocytes in vitro using Renevia. The undifferentiated cells can be seen in FIG. 1A. Differentiation can be seen progressing in FIG. 1B to FIG. 1F, with lipid droplets clearly seen in FIG. 1D through FIG. 1F.

The study enrolled 63 subjects to determine the effectiveness and safety of a lipo-restoring composition comprising autologous adipose derived (SVF) cells delivered to treat HIV-related facial lipoatrophy, in the submalar and perioral areas by:

1) Comparison of the facial volume change between the treatment group and the delayed treatment group; and

2) Assessment of adverse events, including serious adverse events (SAEs), Serious Adverse Device Effects (SADEs) and Unanticipated Serious Adverse Device Effects (USADEs).

Additional informational assessments include:

1) Effectiveness of volume restoration as gauged by the scoring of the MFVDS by independent evaluators and GAIS by subject and independent evaluators using high quality digital photography

2) Subject-reported outcomes including the BIQLI-SP and the Rosenberg Self Esteem Score

3) Skin type using the Fitzpatrick Skin Type Scale

4) Pain as indicated by the VAS pain Score

5) Skin thickness as measured by ultrasound

One effectiveness endpoint of this study was evaluated at Month 6 to determine the effectiveness of a resorbable matrix and SVF in treating HIV-related facial lipoatrophy, which is determined as: The mean change of hemifacial volume at 6 months from baseline as measured by 3D Image Scan, of the “treated” group compared to the that of the “delayed-treatment” control group.

Other endpoints include: For the treatment group, the mean volume retention (percentage) at 6 months post treatment (as calculated by the formula specified infra); there must be no Unanticipated Serious Adverse Device Effects (USADEs); MFVDS: mean change in MFVDS for each group; and GAIS: mean score for each group.

The first three subjects at each site were entered into the Learning Curve Cohort. All other subjects were randomized into 1) a Treatment Group or 2) a Delayed-Treatment Control Group. Within randomized groups, 33 subjects received a single course of treatment of a resorbable matrix and SVF and 30 subjects were assigned to the “delayed-treatment” control group, where treatment will be offered under an extension phase beginning after the initial 6-month follow-up period. The Learning Curve Cohort was not randomized and received the investigational device.

Liposuction was performed under general/local or sedation anesthesia and sterile conditions. Fat tissue was harvested from the abdominal, thighs and/or love handles regions using Microaire lipoaspiration system or other suction assisted lipectomy systems. The use of blunt cannulas 3-4 mm in diameter and 20 cm in length is recommended. The fat was harvested in a GID SVF-1 device (The GID Group Inc, Colorado, USA). The quantity of harvested fat is bound to the amount of abdominal subcutaneous fat available and to facial lipoatrophy severity. Approximately 100-200 mL of dry adipose is collected into each device.

For infiltration, the recommended tumescent solution is a modified Klein's tumescent solution (modifications include Ringer's Lactate, 20 mL lidocaine 1%; and Epinephrine 1:1.000.000. Infiltrated volume equals to the target anticipated procurement volume). Lipoaspirate is harvested with a wet lipoaspiration technique using the aforementioned tumescent solution. To obtain 200 mL dry-lipoaspirate for SVF processing, a minimum of 500 mL tumescence solution should be infiltrated. Other tumescent solution formulas and volume ratio may be utilized, dependent upon the surgeon's criteria.

The adipose tissue is washed and processed under sterile conditions. Once processed the resulting cell pellets can be resuspended and loaded into a 20 mL syringe. An aliquot may be taken for cell quality analysis.

The lipo-restoring composition can be prepared by reconstituting the components and mixing the components according to the instructions presented below. The subject's own lipo-aspirated adipose tissue is processed to obtain autologous SVF cells as delineated above. The liposuction and the injection of the investigational device can occur during the same surgery.

Once the device/cell mixture is ready, the skin is prepared per best practices and up to 5 mL of the resorbable matrix/cell mixture can be drawn into a suitable syringe and then injected subcutaneously into the lipoatrophied areas such as the submalar and perioral regions with a 21- to 30-gauge needle.

Equipment that may be used includes: Incubator and agitator with an agitation capacity of, for example, 150 rpm and constant temperature of about 37° C., Fluid heater (incubator), Cell counter (Chemometec NC-3000, Scepter or equivalent), Centrifuge (Sorvall ST-40 or Heraeus Megafuge 40 w/BioLiner bucket and rotor or equivalent), Digital scale (0.5 g resolution or better), Vacuum source, Micropipettes (10 μL to 100 μL), Stainless steel rack for tubes.

The resorbable matrix is supplied as three vials containing lyophilized powders. The contents of each vial are sterile and nonpyrogenic. Resorbable matrix kits are stored frozen between −25° C. to −10° C. until ready for use. Resorbable matrix kits are removed from freezer and the temperature can be recorded (this step should be done at least one hour prior to the start of surgery). Once resorbable matrix kits are opened, they can be thawed at room temperature for 1 hour.

For reconstitution, the vials are removed from the kit, the plastic tops removed and the tops of the vials wiped with 70% ethanol Using a 3 cc syringe, transfer 2 cc of sterile water for injection into the gelatin vial. A 3 cc syringe is used to transfer 3 cc of sterile water for injection into the Hyaluronan vial. The vials are incubated at 37° C. with orbital shaker at 150 RPM for 30 minutes, or until fully dissolved. Reconstituted solutions may remain at room temperature up to 3 hours before use.

Using established procedures (such as The GID Group's PG 401 and PG 403 or equivalent surgeon's procedures), harvested lipoaspirate is processed to obtain approximately 100-200 cc of dry adipose tissue inside the net of the GID SVF-1 device to obtain a filtrated cellular suspension, free of tissue fragments or fibers. A SVF cell pellet is obtained per GID Group's PG 404. Using a new 20 cc syringe with 14 g spinal needle, the SVF cell pellet is resuspended in approximately 10 cc of lactated ringer's and uptake the resulting SVF suspension. Approximately 1-1.5 mL sample is transferred into a 1.5 ml Eppendorf tube for the cellular quality analysis (which can be conducted after the procedure). The remaining SVF cell suspension is divided equally into two sterile 15 mL conical tubes. The conical tubes are placed in the centrifuge at opposite ends to balance the rotor and centrifuges for 5 minutes at 400×g.

After the SVF cells are fully sedimented via centrifugation, using a sterile needle or a suction pipette, the excess liquid of the upper part of the SVF cell pellet is eliminated in each conical tube (e.g. use the same sterile suction adapter that is used during the isolation of the SVF). As much liquid as possible is removed without disturbing the SVF cell pellet. Using a new 5 cc syringe with 18-20 g needle, the entire contents of the gelatin vial is transferred into the Hyaluronan vial, mixed, and then added to the contents (approximately 5 cc) of the Extralink vial. The vial is swirled or vortexed for 5 seconds to dissolve Extralink to form the Renevia mix. The Renevia mix is transferred using a 3-way valve into a new 5 cc sterile syringe to work inside the field and perform the re-suspension of the first SVF cell pellet. The Renevia mix is transferred into a vessel containing the first SVF cell pellet and swirled by hand until the SVF cells are well suspended in Renevia mix, or if needed, repeatedly drawn into the 5 cc syringe fitted with 14 g spinal needle (3-5 times). The time the resorbable matrix mix is combined with the SVF cell pellet to form the cell/resorbable matrix mix is recorded. The procedure is repeated for the second resorbable matrix mix with the second SVF cell pellet.

The entire volume of cell/resorbable matrix mix (e.g., the lipo-restoring composition) is immediately drawn from one centrifuge tube into a 5 cc syringe fitted with a 14 g spinal needle of 5.5-6 inches. The needle is removed and syringes capped with sterile syringe caps. This is repeated for the second cell/resorbable matrix mix. The total volume of cell/resorbable matrix mix in each syringe is recorded. Using Luer adapter, the cell/resorbable matrix mix is distributed into smaller syringes if desired. Syringes are placed in incubator with agitation. The syringe is maintained at a temperature of 37° C. and in constant motion (100-150 rpm) for at least 10 minutes to allow cell/resorbable matrix mix to begin gelation before injection (the syringes can be left in the sterile tray that previously contained the GID device).

It is preferred that the cell/resorbable matrix mix be injected within 20-30 minutes of mixing, when the gel is becoming viscous (the surgeon can check every 5 to 10 minutes how the consistency of the gel is changing before beginning the injection).

The skin area is washed and disinfected with alcohol or other antiseptic. Immediately prior to the injection, the existing needle is changed with a new sterile 21-30 g needle. The beginning times of the injections and volumes injected into each zone are recorded. Various injection techniques may be employed that vary needle angle, bevel orientation, injection depth, and injection volume. For example, a linear threading method and/or serial punctures may be utilized. The injection area may be massaged if needed to increase conformity with the injection site contours

To be eligible, subjects must exhibit mild to severe HIV-related mid-face volume deficit, as determined by the validated Mid Face Volume Deficit Score (MFVDS) classification system (which is a 6-point grading scale), as well as meeting additional inclusion and exclusion criteria.

Physical Examination

Height, weight, axillar temperature, pulse and blood pressure can be taken and recorded. In addition, at time of enrollment, classification of each subject's skin type will be noted according to the Fitzpatrick Skin Type Scale shown in Table 2.

TABLE 2
Fitzpatrick Skin Type Scale

| Scale | Skin Type Description |
|---|---|
| I | Extremely fair, always burns, never tans |
| II | White, always burns, sometimes tans |
| III | White, sometimes burns, always tans |
| IV | Olive or light brown, rarely burns, always tans |
| V | Brown, never burns |
| VI | Heavily pigmented or black, never burns |

Adverse Event (AE) means any untoward medical occurrence, unintended disease or injury or any untoward clinical signs (including abnormal laboratory finding) in subjects, users, or other persons whether or not related to the Investigational medical device.

Serious Adverse Event (SAE) means An Adverse event that led to a death, injury or permanent impairment to a body structure or a body function, led to a serious deterioration in health of the subject, that either resulted in: a life-threatening illness or injury, or a permanent impairment of a body structure or a body function, or in-patient hospitalization or prolongation of an existing hospitalization or in medical or surgical intervention to prevent life threatening illness, led to fetal distress, fetal death or a congenital abnormality or birth defect.

In the study, no SAEs were reported that were treatment related.

3D digital images can be obtained using an ARTEC 3D Scanner or equivalent and then analyzed. The volume of the area of interest will be recorded at pre-treatment and post-treatment visits at Months 1, 3, 6, 9, 12, 18, and 24. For the untreated control group, similar volume measurements will be made at 1, 3, and 6 months. For untreated control subjects electing to receive treatment, volume changes will be measured as in the original treatment group through the 6-month post treatment extension phase, and extended upon consent to 9, 12, and 18 months after treatment.

For each subject, ultrasound (US) evaluation of the facial lipoatrophy may be performed at baseline and at follow-up visits at 1, 3, and 6 months, with measurements of total cutaneous thickness of the nasogenian area located below the malar bone, in front of the masseter of each cheek can be recorded. Evaluations can be done by the same trained personnel using a digital, multi-frequency, 7.5- to 13-MHz transducer (SonoSite). A detailed US protocol is provided to ensure reproducibility.

The Midface Volume Deficit Score (MFVDS) can be completed by the Evaluating Investigators at pre-treatment and at 1 month, 3 months, 6 months, and at the subject's last follow-up visit. Subjects in both cohorts who participate in the Extension Phase can be evaluated at each follow-up visit (Visits 9 to 12).

At the 1, 3, and 6 month follow-up visits, all subjects and the Evaluating Investigators may assess the subject's level of aesthetic improvement using the Global Aesthetic Improvement Scale (GAIS). Subjects in both cohorts who participate in the Extension Phase can be evaluated at each follow-up visit (Visits 9 to 12). Assessments may be made by comparing sequential pre- and post-treatment digital images. Global Aesthetic Improvement Scale (GAIS) is a commonly used measure of patient satisfaction in dermal filler studies. The GAIS is a relative scale rather than an absolute scale: the subject and evaluator grade the overall improvement by comparing the appearance of the subject at follow-up against the appearance before treatment using pre- and post-treatment photographs.

The subject can complete The Rosenberg Self-Esteem Scale (RSS), which consists of 10 Likert Scale items answered on a 4 point scale from strongly agree to strongly disagree. The change in score from pre-treatment through the follow-up period including the extension phase can be measured.

The Spanish version of the Body Image Quality of Life Inventory (BIQLI-SP) is a self-reported questionnaire that comprises 19 items, 17 of these items are evaluated on a 7-point bipolar scale, from +3 (very positive effect) to 0 (no impact) to −3 (very negative effect). This assessment can be completed by all subjects at pre-treatment, at 1 month, 3 months, 6 months, and at each follow-up visit.

At one month after administration of the lipo-restoring composition, the subject has a Global Aesthetic Improvement Scale (GAIS) as measured by the subject of from about 1 to about 5; from about 2 to about 4; of about 2.8 to about 3.

At three months after administration of the lipo-restoring composition, the subject has a Global Aesthetic Improvement Scale (GAIS) as measured by the subject of from about 1 to about 5; from about 2 to about 4; of about 2.8 to about 3.

At six months after administration of the lipo-restoring composition, the subject has a Global Aesthetic Improvement Scale (GAIS) as measured by the subject of from about 1 to about 5; from about 2 to about 3; of about 2.7 to about 3.

At one month after administration of the lipo-restoring composition, the subject has a Global Aesthetic Improvement Scale (GAIS) as measured by blinded evaluators of from about 2 to about 4.5; from about 3 to about 4; of about 3 to about 3.3.

At three months after administration of the lipo-restoring composition, the subject has a Global Aesthetic Improvement Scale (GAIS) as measured by blinded evaluators of from about 2.5 to about 5; from about 3.5 to about 4.5; of about 4.

At six months after administration of the lipo-restoring composition, the subject has a Global Aesthetic Improvement Scale (GAIS) as measured by blinded evaluators of from about 3 to about 5 or about 4.

At one month after administration of the lipo-restoring composition, the subject has a Rosenberg Self Esteem Score (RSS) measured as a change from baseline of about −2.4 to −0.5; or about −1.4.

At three months after administration of the lipo-restoring composition, the subject has a Rosenberg Self Esteem Score (RSS) measured as a change from baseline of about −3.3 to −0.3, or about −1.8.

At six months after administration of the lipo-restoring composition, the subject has a Rosenberg Self Esteem Score (RSS) measured as a change from baseline of about −3.1 to −0.1, or about −1.6.

At one month after administration of the lipo-restoring composition, the subject has a Body Image Quality of Life Inventory (BIQLI-SP) as measured as a change from baseline of about 12.5 to about 38.0; or about 25.2.

At three months after administration of the lipo-restoring composition, the subject has a Body Image Quality of Life Inventory (BIQLI-SP) as measured as a change from baseline of about 3.8 to about 33.6; or about 18.7.

At six months after administration of the lipo-restoring composition, the subject has a Body Image Quality of Life Inventory (BIQLI-SP) as measured as a change from baseline of about 7.5 to about 36.2; or about 21.8.

Control subjects who elect to be treated will follow the same schedule as treated subjects after the conclusion of the initial investigation.

Randomized Subjects at Month 1, Month 3, and Month 6

One endpoint of this study was evaluated at Month 6 of resorbable matrix and SVF in treating HIV-related facial lipoatrophy: The mean change of hemifacial volume at 6 months from baseline as measured by 3D Image Scan of the “treated” group must be larger (superiority assessment) and statistically significant when compared to that of the “delayed-treatment” control group.

Table 3 shows a summary of the 3D scan volume change data for the control subjects and treatment subjects at 1 month. The data for delayed-treatment group (no treatment administered at this point) is shown in the column labeled “control” and the data for the treatment group is shown in the “procedure.” At 1 month, there were a total of 30 subjects measured in the control group and 32 subjects measured in the treatment group at the 1 month follow-up (FU 1 m). The 3D scan change in volume from the baseline is shown in the third data set (third column). The mean change in volume for the control group was 0 cm³ and the mean change in volume for the treatment group was 6.1 cm³. The treatment effect for the 1 month follow up was calculated as 6.1 (p-value<0.001).

TABLE 3
3D Scan Volume Change from Screening at 1 Month

| | Control (n = 30) | Procedure (n = 32) |
|---|---|---|
| Follow-up at 1 month - | 0 | 6.1 |
| change from screening | | |
| P-value | | <.001 |
| Treatment Effect (mean) | | 6.1 |
| P-value | | <.001 |

Table 4 shows a summary of the 3D scan volume change data for the control subjects and treatment subjects at 3 months. The mean change in volume for the control group remained at 0 cm³ and the mean change in volume for the treatment group was 5.6 cm³ (n=30; p-value<0.001). The treatment effect showed a statistically significant value of 5.6 cm³ (p-value<0.001).

TABLE 4
3D Scan Volume Change from Screening at 3 Months

| | Control (n = 25) | Procedure (n = 30) |
|---|---|---|
| Follow-up at 3 months | 0 | 5.6 |
| P-value | | <.001 |
| Treatment Effect (mean) | | 5.6 |
| P-value | | <.001 |

Table 5 shows a summary of the 3D scan volume change data for the control subjects and treatment subjects at 6 months. The mean change in volume for the control group dropped to −0.1 cm³ and the mean change in volume for the treatment group was 5.1 cm³ (n=26; p-value<0.001) from baseline (screening). The treatment effect showed a statistically significant value of 5.2 cm³ (p-value<0.001) when the differences in the mean 3D volume changes for the treatment and control groups were compared.

TABLE 5
3D Scan Volume Change from Screening at 6 Months

| | Control (n = 21) | Procedure (n = 26) |
|---|---|---|
| Follow-up at 6 months | −0.1 | 5.1 |
| P-value | | <.001 |
| Treatment Effect (mean) | | 5.2 |
| P-value | | <.001 |

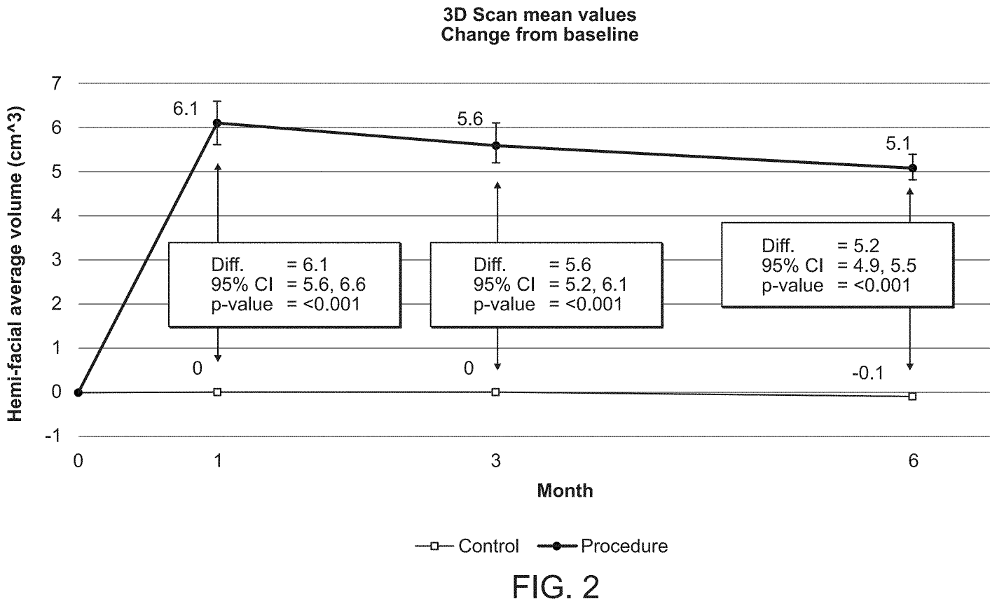

The mean Hemifacial Incremental Volume for the treatment group (resorbable matrix (Renevia) and SVF) and the control group (no treatment) is shown in FIG. 2. These results demonstrate that the study achieved one of its endpoints, namely the mean change in hemifacial volume at 6 months for treated subjects versus controls as measured by 3D image assessment. Subjects were administered about 10 cc (a mean of 9.7 cc and a median of 10 cc) hemifacially. Said in another way, half of the amount was administered to each side of the face. These results demonstrate that an average volume (mean and median) of 5.1 cc was retained at 6 months. Because it has been shown that Renevia can be reabsorbed in vivo after approximately 6 months, these results indicate that the administered adipose derived cells have engrafted and are proliferating.

At 3 months, from about 54.2% to about 149.5% retention of volume was demonstrated, as compared to one month after administration. A mean of about 93.3% and a median of about 90.7% retention of volume administered was demonstrated, as compared to one month after administration. From about 83.1% to about 103.1% of administered volume was retained at 3 months post administration, as compared to one month after administration.

At 6 months, from about 43.3% to about 115.6% retention of volume was demonstrated as compared to one month after administration. A mean of 85.7% and a median of 89.2% retention was demonstrated, as compared to one month after administration. From about 43.3% to about 115.6% of the administered volume was retained 6 months after administration, as compared to one month after administration. At 6 months after administration, about 100% retained volume was demonstrated in a subject.

At 1 month, from about 82% to about 182% retention of volume was demonstrated as compared to the administered volume. At 1 month, from about 104% to about 134% retention of volume was demonstrated as compared to the administered volume. At 1 month after administration, about 122% retained volume was demonstrated in a subject.

At 3 months, from about 88% to about 192% retention of volume was demonstrated as compared to the administered volume. At 3 months, from about 98% to about 118% retention of volume was demonstrated as compared to the administered volume. At 3 months after administration, about 112% retained volume was demonstrated in a subject.

At 6 months, from about 74% to about 136% retention of volume was demonstrated as compared to the administered volume. At 6 months, from about 92% to about 108% retention of volume was demonstrated as compared to the administered volume. At 6 months after administration, about 100% retained volume was demonstrated in a subject.

On average, about 93% of the administered volume was retained 12 months after administration. At 12 months, from about 84% to about 102% retention of volume was demonstrated in subjects. At 12 months, from about 4.29 cc to about 5.10 cc of volume was retained.

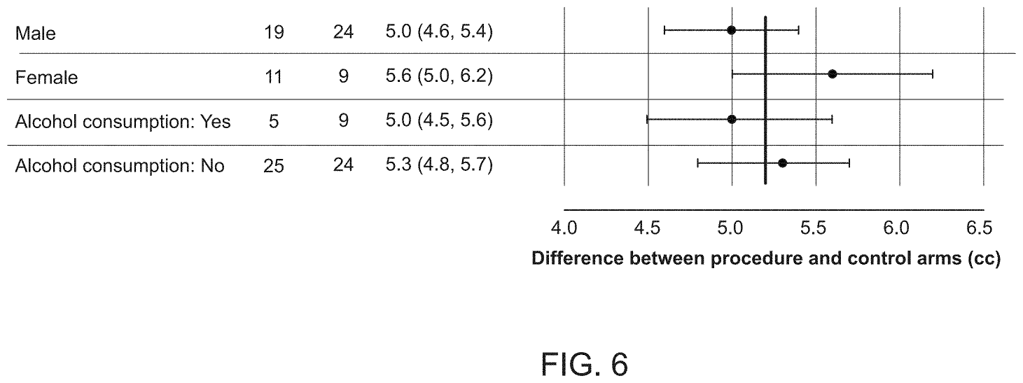

Female subjects may respond better than male subjects. FIG. 6 shows that females administered about 10 cc of the lipo-restoring composition retained more average volume than male subjects at 6 months.

Subjects that do not consume alcohol may respond better than subjects that do consume alcohol. FIG. 6 shows that subjects who did not consume alcohol that were administered about 10 cc of the lipo-restoring composition retained more average volume than subjects that did consume alcohol.

### Example 2

Enrollment additionally included seven (7) non-randomized subjects at each site for training purposes. The subjects are not randomized and are treated to allow the physician to gain experience with the protocol. Incremental volume was determined by the same 3D photographic volumetric assessment methodology as was used with randomized subjects, as described above.

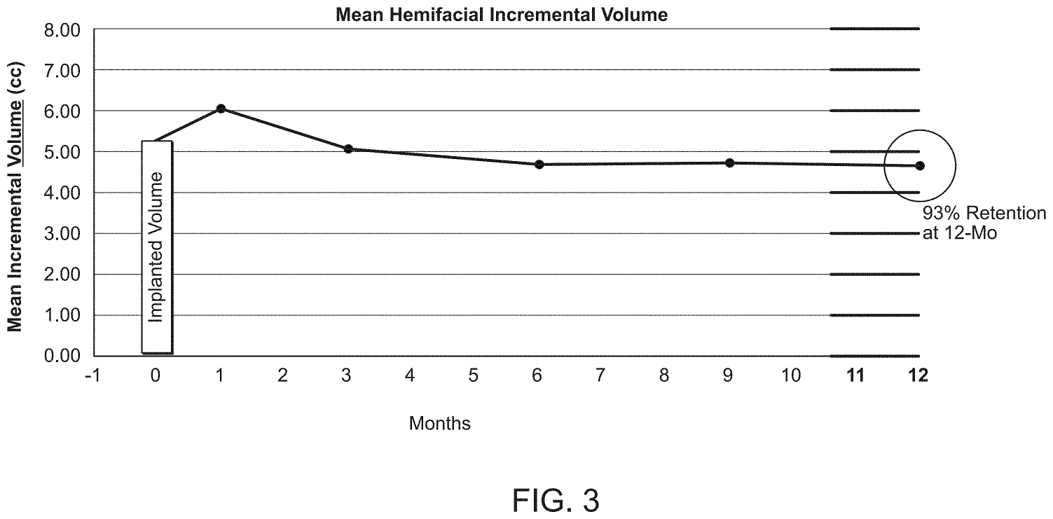

The mean hemifacial incremental volume was determined for 7 subjects at baseline (pre-treatment), 1 month, 3 month, 6 month, 9 month and 12 month time points. The results are presented in FIG. 3 and illustrate that an average of 93% of the volume is retained by the 12-month time point.

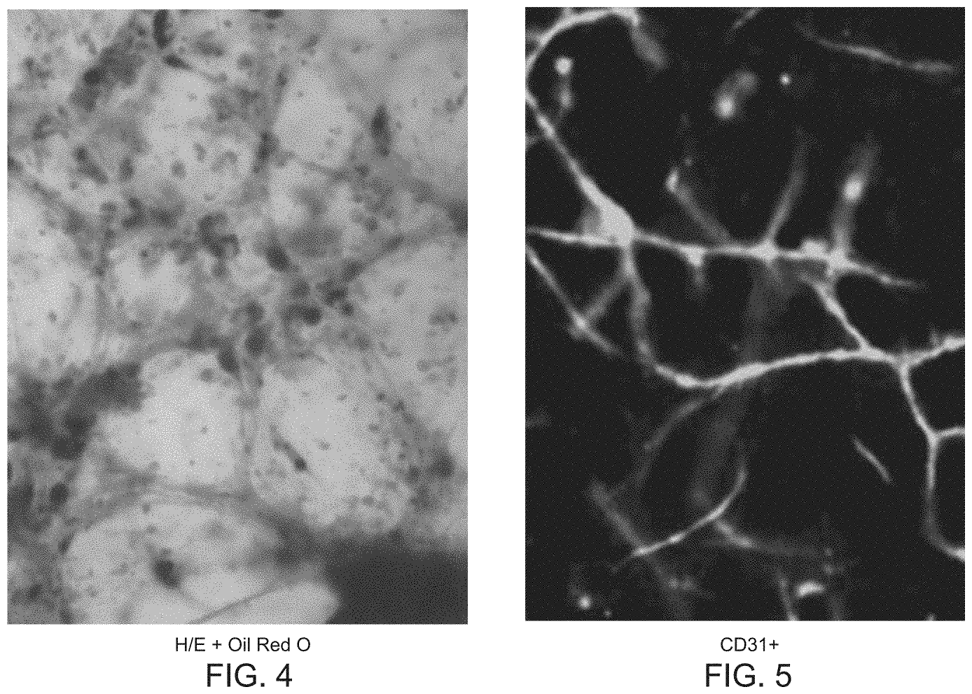

In addition, biopsies were taken from subjects that developed hematomas and the biopsy samples were analyzed for adipocyte formation and evidence of vascularization. The results are presented in FIG. 4 and FIG. 5. FIG. 4 shows an Oil Red O (H/E+Oil Red O) stained histological section of the one of the biopsies taken from a subject. The biopsy was taken between the 6 month and 12 month follow-up. The staining indicates the formation of adipocytes, as can be seen by the large lipid deposits. In FIG. 5, evidence of vascularization can be seen within the biopsy sample stained with CD31. This demonstrates that the implant is becoming vascularized. This is further evidence that the cells in the lipo-restoring composition attach, proliferate, and differentiate into adipocytes.

### Example 3

### Subjects Retained Most of the Administered Volume at 18 Months

Subjects with HIV-associated lipoatrophy received approximately 5 cc of resorbable matrix and SVF in each side of the face (hemifacial). All transplants were well tolerated and there were no device-related serious adverse events. The primary endpoint was the change in hemifacial volume at six months in the treated subjects compared to subjects in the delayed treatment group as measured by 3-D photographic volumetric assessment. However, long-term data points on volume retention were also collected.

Table 6 shows the average (mean) volume measured through 18 months. Facial volume change from baseline was further measured in the treatment subjects by three-dimensional (3D) image scans at 9, 12, and 18 months according to methods described in Example 1. The mean change in volume from baseline (screening) for the treatment group was 4.1 cm³ (n=21) at 9 months after treatment. The mean change in volume from baseline for the treatment group at 12 months post treatment was 3.5 cm³ (n=15). And the mean change in volume from baseline for the treatment group at 18 months after treatment was 3.2 cm³ (n=5).

TABLE 6
Average 3D Scan Volume Change from
Screening at 6, 9, 12 and 18 Months

| | 6 Months | 9 Months | 12 Months | 18 Months |
|---|---|---|---|---|
| n | 28 | 21 | 15 | 5 |
| Mean volume measured | 5.1 | 4.1 | 3.5 | 3.2 |
| Percent of retention | 100% | 82% | 70% | 64% |

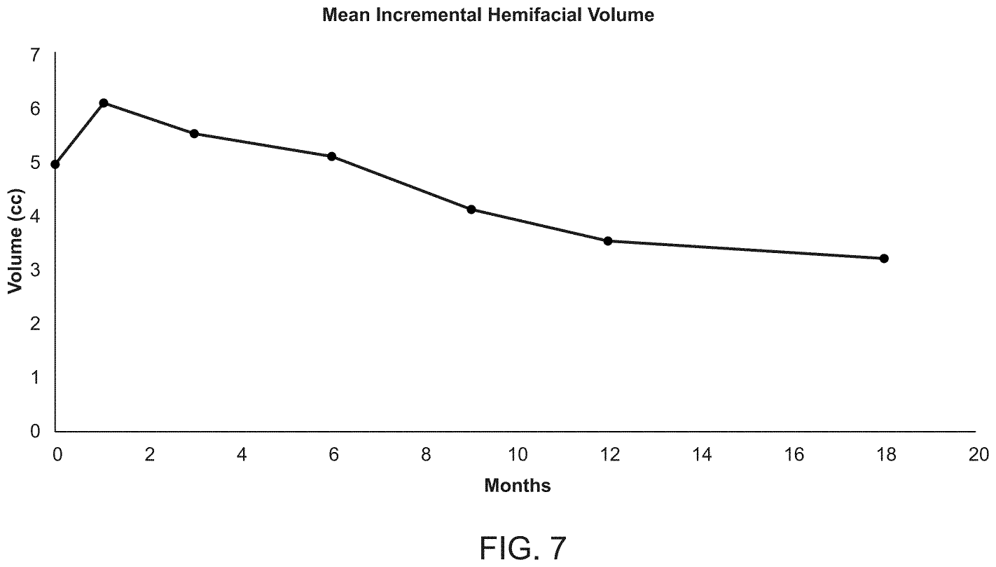

At 9 months, at least about 82% (ranges were seen from about 76% to about 90%; from about 40% to about 92%; from about 82% to about 88% with 21 patients at 9 months) retention of volume was demonstrated. At 12 months, at least about 70% (ranges were seen from about 62% to about 84%; from about 30% to about 90%; from about 70% to about 76%, with 15 patients at 12 months) retention of volume was demonstrated. And at 18 months, at least about 64% (ranges were seen from about 56% to about 76%; from about 46% to about 78%; from about 64% to about 68% with only 5 patients at 18 months) retention of volume was demonstrated. FIG. 7 shows the 3D scan percentages of retention for follow-up months 1, 3, 6, 9, 12 and 18 for the treatment group. Because it has been shown that Renevia can be reabsorbed in vivo after approximately 6 months, these results further indicate that the administered adipose derived cells have engrafted and are proliferating.

Loss of fat from the face can be one of the most stigmatizing signs of HIV-associated lipoatrophy. Although 3D scan volume was measured through 18 months, the results for the retained volume are surprising. Fat transfer alone is reported to have unpredictable outcomes and generally have a short duration of effect, with retention rates of approximately 40% at 12 months in a non-HIV patient population. Subjects treated with the resorbable matrix and SVF as described herein showed that about 70% of the transplanted volume was retained at 12 months, which 75% greater than fat transfers.

### Example 4

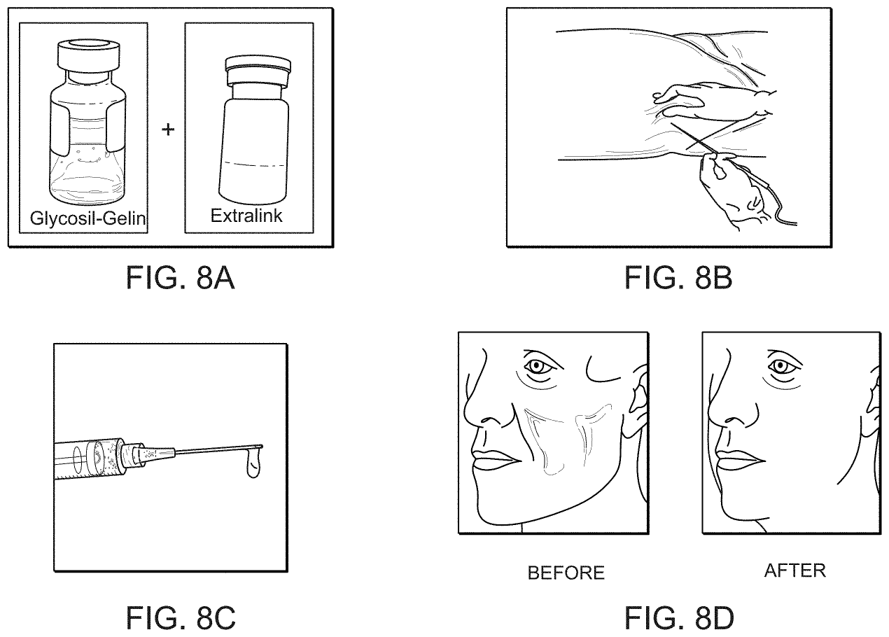

FIG. 8A through FIG. 8D illustrate use of a resorbable matrix with fat. FIG. 8A shows an example of the hyaluronan/gelatin component of a resorbable matrix and a linking agent (e.g., Extralink). If the components are frozen, they can be removed from the freezer and thawed for about 90 minutes prior to treatment. Hyaluronan and gelatin component flows like water when thawed. FIG. 8B shows an illustration of fat tissue being harvested by liposuction. While the components are thawing, the clinician can obtain fat from the patient via a surgical practice known to one of skill in the art. The autologous fat particles are processed to preserve the function and characteristics of whole adipose tissue. Unlike isolated cells that are stripped from their extracellular matrix through enzyme processing, the fat tissue that is added to the resorbable matrix is only mechanically disrupted and remains incorporated in an extracellular matrix.

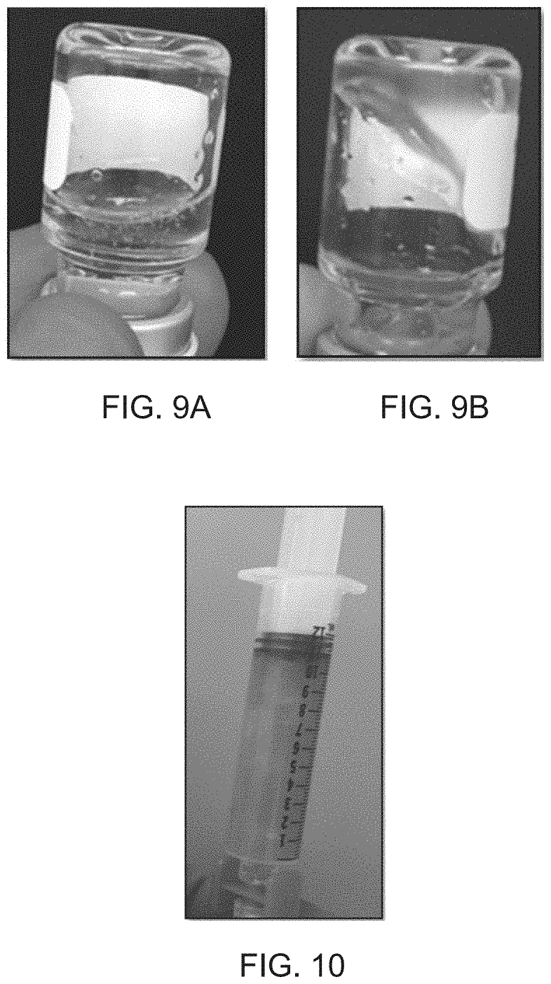

As an example of a preparation of a particular resorbable matrix appropriate for use with fat, thawed Hyaluronan and gelatin may be combined aseptically with Extralink to form the Renevia hydrogel. Cross-linking of the components is achieved through the Michael addition of the thiol moiety on the macromolecules to the double bonds at each end of the cross-linker. In this example, the reaction proceeds without byproducts or changes in temperature or pH, and occurs in situ. During the cross-linking process, the molecules interact with each other to create the three-dimensional hydrogel matrix. FIG. 8D shows what the Hyaluronan and gelatin component looks like when Extralink is added and allowed to gel, according to some embodiments. The gelated product becomes a soft scaffold that is retained at bottom of vial after upending. FIG. 9A shows the pre-gelled Renevia hydrogel, according to certain embodiments. FIG. 9B shows the Renevia hydrogel after gelation, according to certain embodiments.

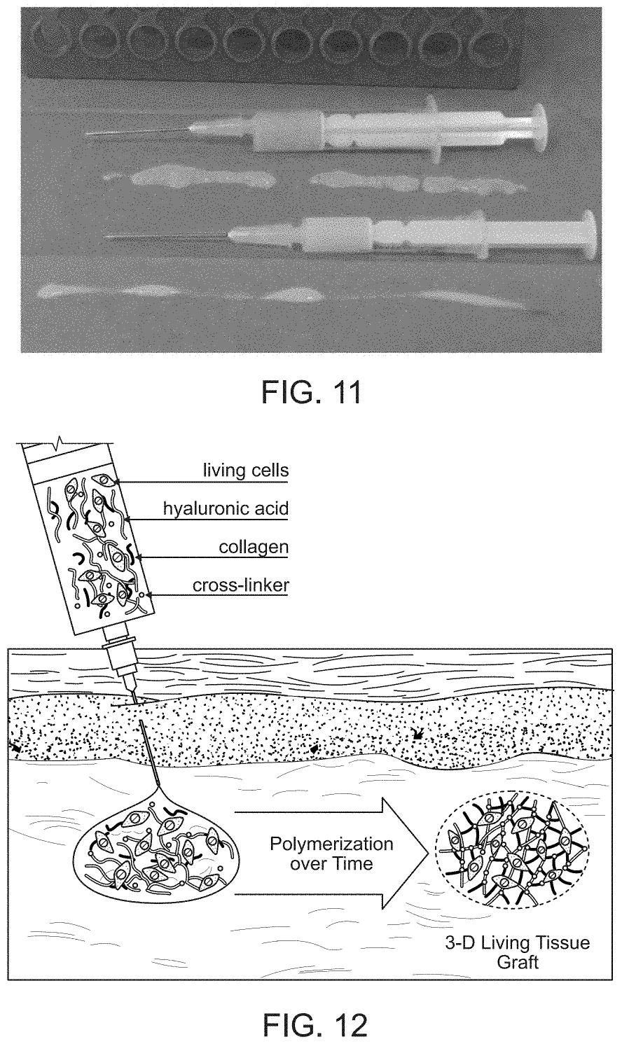

After combination of the Renevia components (as an example of a resorbable matrix), the combination may be mixed with fat (prior to gelation), and drawn into a syringe and an appropriately gauged needle. The hydrogel-fat mixture is allowed to cure for about 10 minutes after the Renevia components have been combined. Homogeneity can be ensured by regular inversion or rotation of the syringe. Gel consistency can be checked for signs of gelation. In this step, the fat tissue particles become physically encapsulated/entangled within the forming hydrogel. FIG. 10 shows the yellow fat parcels evenly distributed throughout the gel. The reddish color come from blood components in the fat sample. Once gelation starts, the hydrogel-fat mixture can be injected within about 10 minutes via established surgical procedures (various techniques known to one of skill in the art for injecting dermal fillers or fat may be employed). FIG. 11 demonstrates what fat alone (bottom syringe) and hydrogel+fat (top syringe) looks like when extruded from a syringe. The hydrogel-fat mixture gels after extrusion, as shown in FIG. 11.

### Example 5

In the present example, methods for lipoaspiration and SVF isolation of subcutaneous fat from a pig, evaluation of parameters for transplantation (Coleman vs bolus, number cells required/ml, site for transplantation), and use of a pig model to establish kinetics of new fat formation vs. standard of care (fat transfer) are presented.

Stromal vascular fraction (SVF) cells are the non-adipose cells (undifferentiated and differentiated) derived from a lipoaspirate. In some embodiments, Adipose Derived Stem Cells (ADSC), the SVF cellular fraction, which adheres to tissue culture plastic in traditional media, can be used in Autologous Stem Cell Transfer (ASCT) methods. SVF is an excellent source of cells for adipose tissue regeneration. Adipose-derived cells are sturdy enough to be harvested using liposuction, and the SVF contains precursor cells to ensure the requisite vasculature infrastructure is formed simultaneously with adipocyte differentiation. The SVF is abundant in three key stem and progenitor cell types: multipotent stem cells, preadipocytes, and endothelial precursor cells.

While the SVF contains the requisite cells to restore subcutaneous fat, successful resolution of contour defects is enhanced by their delivery in a matrix that creates a scaffold to form durable 3D tissue constructs. The natural contact inhibition exhibited by the SVF cells limits their proliferation and efficacy to resolve such defects. Although not an inclusive list, examples of useful characteristics of a cell delivery matrix are: (1) enhance the survival of implanted cells so that cells are able to differentiate, self-assemble, and eventually engraft; (2) localize the cells to the site of implantation; (3) protect the cells by providing a lifelike, encapsulated microenvironment; and (4) provide a volume into which implanted cells can grow by preventing contact inhibition.

Examples of biocompatible matrices are hydrogels. An example of a hydrogel with the characteristics described above includes hyaluronic acid based hydrogels. Hydrogels that are capable of crosslinking in situ to enable cells to be readily mixed and delivered by injection prior to gelation are also useful for the delivery of tissue or cells, such as SVF, as shown in FIG. 12. In the present example, the resorbable matrix can provide cellular attachment sites to prevent SVF cells from undergoing programmed cell death that occurs in anchorage-dependent cells and can provide a cellular microenvironment which significantly improves cellular survival and engraftment.

While there are immunodeficient rodent models to study fat transfer (Paik K J. et al., Plast Reconstr Surg. 2015), a competent immune system is helpful for a robust healing/regenerative process. The Gottingen minipig represents a useful model due to its similarity to human fat in terms of adipose-related gene expression (Cirera S. et al., Anim Genet. 2014) and for the ability to extract sufficient amounts of tissue (100-200 g of fat per minipig corresponding to 50-100 MM (million) SVF cells total per minipig).

Liposuction can be successfully performed on pigs and SVF isolated from the recovered fat tissue. One embodiment of the present method establishes the yield and quality of the resulting SVF from Gottingen minipigs.

In one embodiment, duplicate 1-5 cc injections can require approx. 10 MM SVF cells/ml, and a minimum yield of 100 MM SVF cells/animal. Harvested adipose tissue can be processed and SVF cells isolated. The SVF cells can be quantitated by nuclear staining using propidium iodide and ADSCs are isolated by passage onto plastic plates. The resulting cells may be characterized for their ability to both proliferate (using Alamar Blue analysis and Live dead staining) and differentiate into adipocytes on a resorbable matrix (using Adipogenic differentiation kits). The surface expression of CD29, CD44, CD90 and MHC I (or HLA I) (characteristic of ADSCs) and CD4a, CD31, CD45 and MHC II (or HLA II) (characteristic of contaminating cells) can be analyzed by flow cytometry (Chen Y J et al., J. Vis. Exp. 2016).

This embodiment can demonstrate that 1) at least 100 MM SVF cells can be isolated per minipig, and 2) ADSCs from the SVF double every 7 days, are capable of differentiation into adipocytes, and express the expected markers of ADSCs.

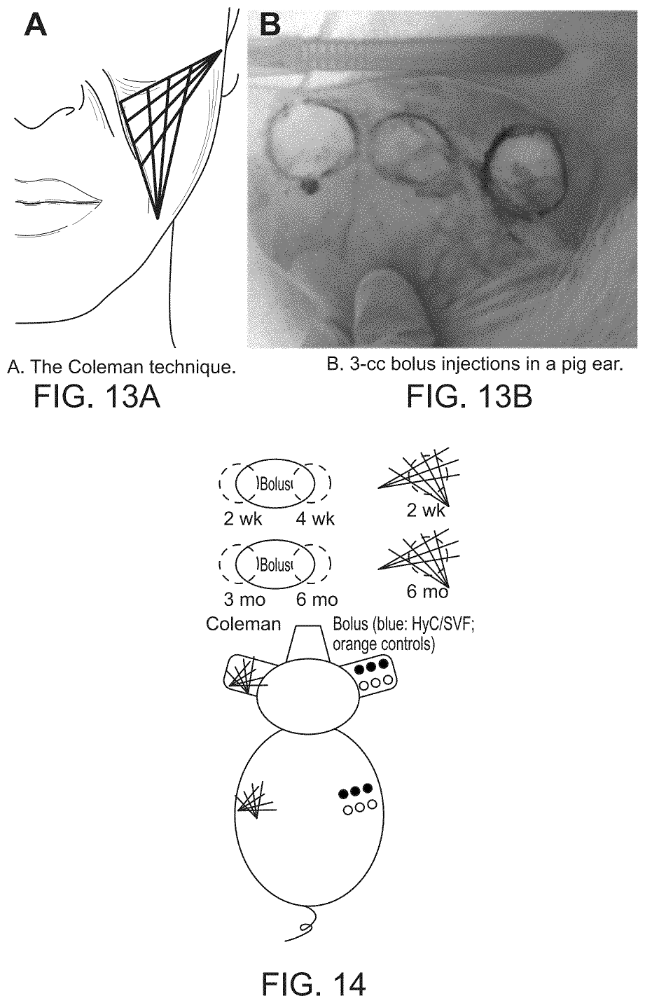

Transplanted SVF are sustained with nutrients and thus, an injection volume that allows rapid nutrient diffusion is beneficial for fat formation and to prevent cellular necrosis. Yet for analysis, the thin tracks containing autologous unlabeled SVF cells can be difficult to discern. Therefore, bolus implants of between 1 cc and 3 cc can be evaluated, as shown in FIG. 13B. Preferably, the area to be injected is not subject to liposuction. For example, the flank or the ear may be used.

In one embodiment, to evaluate injection location, SVF cells are isolated and combined with a resorbable matrix as described above to a final concentration of 10 MM SVF cells/ml followed by subcutaneous injection. A baseline volumetric measurement of the area is taken using a 3D scanner (Artek, Inc, Palo Alto, Calif.). Four, 9-week-old female Gottingen minipigs undergo liposuction and SVF is prepared and quantitated. Each animal may receive 5 cc in the left ear and flank in a 1.5-2″ diameter (17 gauge needle cannula length). On the right side, 3×1 cc injections can be placed into the ear and flank (FIG. 14). As controls, a resorbable matrix and SVF alone in 1× PBS and fat alone may be used as a positive control. Injection sites can be tattooed and biopsies taken from injected areas post-injection (depending on method; FIG. 14) and fixed in formalin. The Coleman technique can also be used, as demonstrated in FIG. 13A.

Implants undergo 3D volumetric measurement prior to biopsy and harvested tissues can be sectioned for histological analysis: H&E staining, Oil Red O (stains adipocytes), and cresyl violet (stains undegraded hyaluronan) may be used.

The methods described can demonstrate that injected SVF or fat can be easily identified within the resorbable matrix, and the implant does not change shape or position with the animal activity. Therefore, the method (Coleman or bolus) can demonstrate fat formation.

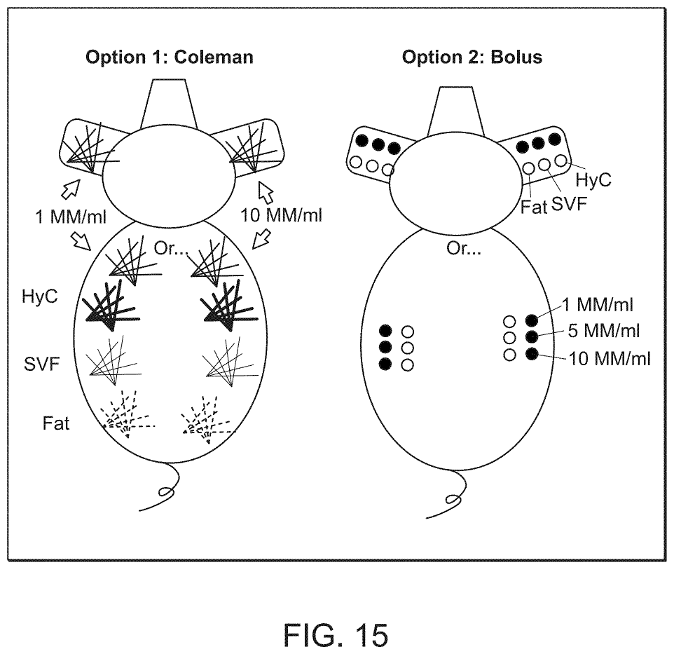

In another embodiment, initial 3D volumetric measurements can be taken (Artek, Inc, Palo Alto Calif.), injection site tattooed, and 3 doses of Renevia delivered subcutaneously. In another embodiment, the Coleman technique can be used and each animal (n=4) may receive either a 5 cc 1 MM SVF cells/ml or a 5 cc 10 MM SVF cells/ml subcutaneous implantation in the left and right ear and flank (FIG. 15, left). In another embodiment, the bolus implantation can be used and each animal (n=4) may receive 1 cc subcutaneous injections on both ears and flanks (3 per side for a total of 6); injections may contain either 1 MM, 5 MM, or 10 MM SVF cells. resorbable matrix or SVF alone in 1× PBS and fat alone can be used as controls (FIG. 15).

3D volumetric measurements and biopsies may be taken at 1, 3, 6 months post-injection. Harvested tissues are sectioned for histological analysis and H&E, specific stains for adipocytes (e.g. Oil Red O), and undegraded hyaluronan from HA hydrogels (cresyl violet) may be used as stains.

The methods described can demonstrate whether more adipose tissue is formed or retained compared to controls at the 6-month time point based on comparison of the resulting volume and density of Oil Red staining cells per section. Additionally, the treatment should maintain a localized presence and have no deleterious effects on surrounding tissues and can be demonstrated by the methods described.

Standardized and reproducible procedures can lead to more reliable improvement of body contour as well as a longer lasting effect compared to traditional fat transfer.

Resorbable matrices in combination with a subject's own SVF to regenerate fat tissue can dramatically change the reproducibility of producing more permanent restoration of a subject's contour defects and obviates the need for repeat injections currently required for whole fat transfer.

From the description herein, it will be appreciated that that the present disclosure encompasses multiple embodiments which include, but are not limited to, the following:

A method of correcting a soft tissue defect caused by lipoatrophy in a subject, the method comprising restoring tissue to the lipoatrophic area.

A method of, one or more of, slowing the progression of lipoatrophy, slowing the progression of facial lipoatrophy, preventing facial volume decrease, restoring facial volume, increasing facial volume for greater than 6 months, or treating subcutaneous facial lipoatrophy defects in a subject, comprising: administering a lipo-restoring composition to the face, wherein the lipo-restoring composition comprises a combination of a resorbable matrix and adipose derived cells.

The method of any previous embodiment, wherein facial lipoatrophy comprises one or more of: mid-face volume deficit, mild to severe submalar volume deficit, mild to severe perioral volume deficit, or subcutaneous contour defects.

The method of any previous embodiment, wherein the defects arise from HIV infection or HAART treatment.

The method of any previous embodiment, wherein the defects are from one or more of: secondary to congenital abnormalities, trauma, surgical resection, aging processes, and disease.

The method of any previous embodiment, wherein the defects arise from infection, diabetes, auto immune disease, acquired generalized lipodystrophy (AGL), Lawrence syndrome, acquired partial lipodystrophy (APL), progressive lipodystrophy, Barraquer-Simons syndrome, injury, weight loss, repeated injection site, or localized pressure.

The method of any previous embodiment, wherein an infection comprises one or more of measles, pneumonia, infectious mononucleosis, or hepatitis.

The method of any previous embodiment, wherein after administration the adipose derived cells attach, proliferate, and differentiate into adipocytes.

The method of any previous embodiment, wherein after administration the adipose derived cells attach.

The method of any previous embodiment, wherein after administration the adipose derived cells proliferate.

The method of any previous embodiment, wherein after administration the adipose derived cells differentiate into adipocytes.

The method of any previous embodiment, wherein the adipose derived cells comprise one or more of: autologous adipose derived cells, stromal vascular cells, stromal vascular fraction, multipotent stem cells, pre-adipocytes, and endothelial precursor cells.

The method of any previous embodiment, wherein the adipose derived cells are isolated from fat tissue harvested from the subject.

The method of any previous embodiment, wherein the subject is the same subject that receives the lipo-restoring composition or is an unrelated subject.

The method of any previous embodiment, wherein the fat cells are obtained by liposuction.

The method of any previous embodiment, wherein about 50-500 mL of dry adipose is collected from the liposuction.

The method of any previous embodiment, wherein the adipose is washed and processed to obtain a cell pellet.

The method of any previous embodiment, wherein the facial volume is measured against a baseline.

The method of any previous embodiment, wherein the baseline comprises a measurement made prior to administration.

The method of any previous embodiment, wherein the facial volume change is for greater than 9 months.

The method of any previous embodiment, wherein the facial volume change is for greater than 12 months.

The method of any previous embodiment, wherein the facial volume change is for greater than 18 months.

The method of any previous embodiment, wherein a female subject responds better than a male subject, wherein responding better comprises having a greater average facial volume increase at 6 months than a male.

The method of any previous embodiment, wherein a subject that does not consume alcohol responds better than a subject that does, wherein responding better comprises having a greater average facial volume increase at 6 months than a subject that does consume alcohol.

The method of any previous embodiment, wherein at 3 months a subject has from about 54.2 to about 149.5% retention of volume.

The method of any previous embodiment, wherein at 3 months a subject has about 93.3% retention of volume.

The method of any previous embodiment, wherein at 3 months a subject has about 90.7% retention of volume.

The method of any previous embodiment, wherein at 3 months a subject has from about 83.1 to about 103.1% volume retained.

The method of any previous embodiment, wherein at 6 months a subject has from about 43.3 to about 115.6% retention of volume.

The method of any previous embodiment, wherein at 6 months, about 85.7% of the volume was retained.

The method of any previous embodiment, wherein at 6 months about 89.2% volume was retained.

The method of any previous embodiment, wherein at 6 months a subject has from about 43.3 to about 115.6% volume retained.

The method of any previous embodiment, wherein at 9 months, about 82% of the volume was retained.

The method of any previous embodiment, wherein at 9 months, about 88% of the volume was retained.

The method of any previous embodiment, wherein at 9 months about 76% to about 90%; from about 40% to about 92%; or from about 82% to about 88% volume was retained.

The method of any previous embodiment, wherein at 12 months, about 70% of the volume was retained.

The method of any previous embodiment, wherein at 12 months at least about 88% of the volume was retained

The method of any previous embodiment, wherein at 12 months from about 62% to about 84%; from about 30% to about 90%; or from about 70% to about 76% of the volume was retained.

The method of any previous embodiment, wherein at 18 months, about 64% of the volume was retained.

The method of any previous embodiment, wherein at 18 months at least about 68% of the volume was retained

The method of any previous embodiment, wherein at 18 months from about 56% to about 76%; from about 46% to about 78%; or from about 64% to about 68% of the volume was retained.

The method of any previous embodiment, wherein at 12 months about 93% of the administered volume was retained.

The method of any previous embodiment, wherein at 12 months after administration from about 84% to about 102% retention of volume was demonstrated in subjects.

The method of any previous embodiment, wherein at one month after administration of the lipo-restoring composition, the subject has a Global Aesthetic Improvement Scale (GAIS) as measured by the subject of from about 1 to about 5; from about 2 to about 4; of about 2.8 to about 3.

The method of any previous embodiment, wherein at two months after administration of the lipo-restoring composition, the subject has a Global Aesthetic Improvement Scale (GAIS) as measured by the subject of from about 1 to about 5; from about 2 to about 4; of about 2.8 to about 3.

The method of any previous embodiment, wherein at six months after administration of the lipo-restoring composition, the subject has a Global Aesthetic Improvement Scale (GAIS) as measured by the subject of from about 1 to about 5; from about 2 to about 3; of about 2.7 to about 3.

The method of any previous embodiment, wherein at one month after administration of the lipo-restoring composition, the subject has a Global Aesthetic Improvement Scale (GAIS) as measured by blinded evaluators of from about 2 to about 4.5; from about 3 to about 4; of about 3 to about 3.3.

The method of any previous embodiment, wherein at two months after administration of the lipo-restoring composition, the subject has a Global Aesthetic Improvement Scale (GAIS) as measured by blinded evaluators of from about 2.5 to about 5; from about 3.5 to about 4.5; of about 4.

The method of any previous embodiment, wherein at six months after administration of the lipo-restoring composition, the subject has a Global Aesthetic Improvement Scale (GAIS) as measured by blinded evaluators of from about 3 to about 5 or about 4.

The method of any previous embodiment, wherein at one month after administration of the lipo-restoring composition, the subject has a Rosenberg Self Esteem Score (RSS) measured as a change from baseline of about −2.4 to −0.5; or about −1.4.

The method of any previous embodiment, wherein at three months after administration of the lipo-restoring composition, the subject has a Rosenberg Self Esteem Score (RSS) measured as a change from baseline of about −3.3 to −0.3, or about −1.8.

The method of any previous embodiment, wherein at six months after administration of the lipo-restoring composition, the subject has a Rosenberg Self Esteem Score (RSS) measured as a change from baseline of about −3.1 to −0.1, or about −1.6.

The method of any previous embodiment, wherein at one month after administration of the lipo-restoring composition, the subject has a Body Image Quality of Life Inventory (BIQLI-SP) as measured as a change from baseline of about 12.5 to about 38.0; or about 25.2.

The method of any previous embodiment, wherein at three months after administration of the lipo-restoring composition, the subject has a Body Image Quality of Life Inventory (BIQLI-SP) as measured as a change from baseline of about 3.8 to about 33.6; or about 18.7.

The method of any previous embodiment, wherein at six months after administration of the lipo-restoring composition, the subject has a Body Image Quality of Life Inventory (BIQLI-SP) as measured as a change from baseline of about 7.5 to about 36.2; or about 21.8.

The method of any previous embodiment, wherein the resorbable matrix comprises a hydrogel.

The method of any previous embodiment, wherein the hydrogel comprises thiol-modified hyaluronan, thiol-modified gelatin, and polyethylene glycol diacrylate (PEGDA).

The method of any previous embodiment, wherein the thiol-modified hyaluronan, thiol-modified gelatin, and polyethylene glycol diacrylate (PEGDA) are in a lyophilized format.

The method of any previous embodiment, wherein the hydrogel is made by a method comprising: (a) reconstituting the thiol-modified hyaluronan, thiol-modified gelatin, and polyethylene glycol diacrylate (PEGDA); and (b) mixing the thiol-modified hyaluronan, thiol-modified gelatin, and polyethylene glycol diacrylate (PEGDA) together.

The method of any previous embodiment, wherein the hydrogel is hyaluronan based with a hyaluronan concentration of between about 1 mg/mL to about 20 mg/mL.

The method of any previous embodiment, wherein the hydrogel is hyaluronan based with a hyaluronan concentration of between about 3 mg/mL to about 5 mg/mL.

The method of any previous embodiment, wherein the hydrogel further comprises gelatin at a concentration of between about 1 mg/mL to about 20 mg/mL.

The method of any previous embodiment, wherein the hydrogel further comprises gelatin at a concentration of between about 3 mg/mL to about 5 mg/mL.

The method of any previous embodiment, wherein the hydrogel has a weight ratio of the hyaluronan to the gelatin of between about 1:1 to about 10:1; between about 1:1 to about 1:10; about 1:1.5; about 1.5:1; about 1:2; about 2:1; or from between about 0.5:5 to about 5:0.5.

The method of any previous embodiment, wherein the hydrogel is made by a method comprising: (1) reacting a first thiolated polymer with GSSG; and (2) adding a second thiolated polymer to the reaction, thereby forming a hydrogel comprising the first and second thiolated polymers, wherein GSSG is not crosslinked to a polymer.

The method of any previous embodiment, wherein the first thiolated polymer is thiolated carboxymethylated hyaluronan and wherein the second thiolated polymer is thiolated gelatin.

The method of any previous embodiment, wherein the resorbable matrix has a storage modulus value of between about 1 Pa and 1,000 Pa.

The method of any previous embodiment, wherein the lipo-restoring composition is administered while the lipo-restoring composition has a storage modulus of between about 0.1 Pa and about 10 Pa and wherein the lipo-restoring composition continues to cure in situ to between about 20 Pa to about 150 Pa.

The method of any previous embodiment, wherein the resorbable matrix comprises SLF.

The method of any previous embodiment, wherein SLF is made by a method comprising:

(a) thawing a combination of thiol-modified hyaluronan and thiol-modified gelatin at a temperature of approximately 35° C. or greater; and

(b) adding polyethylene glycol diacrylate (PEGDA) to the thawed combination of thiol-modified hyaluronan and thiol-modified gelatin.

The method of any previous embodiment, wherein the lipo-restoring composition is made by a method comprising: suspending the adipose derived cells in the resorbable matrix.

The method of any previous embodiment, wherein the suspending is mixing.

The method of any previous embodiment, wherein 5 ml of the lipo-restoring composition is administered to the face.

The method of any previous embodiment, wherein between about 1 ml and about 5 ml of the lipo-restoring composition is administered to the face.

The method of any previous embodiment, wherein between about 5 ml and about 10 ml of the lipo-restoring composition is administered to the face.

The method of any previous embodiment, wherein between about 1 ml and about 20 ml of the lipo-restoring composition is administered to the face.

The method of any previous embodiment, wherein between about 1 ml and about 40 ml of the lipo-restoring composition is administered to the face.

The method of any previous embodiment, wherein between about 5 ml and about 15 ml of the lipo-restoring composition is administered to each side of the face.

The method of any previous embodiment, wherein the lipo-restoring composition comprises an implant.

The method of any previous embodiment, wherein the adipose derived cells engraft after administration.

The method of any previous embodiment, wherein the engrafted cells vascularize.

The method of any previous embodiment, wherein the adipose derived cells progress to lipocytes after administration.

A method of one or more of slowing the progression of facial lipoatrophy, preventing facial volume decrease, restoring facial volume, increasing facial volume for greater than 6 months, or treating subcutaneous facial lipoatrophy defects in a subject, comprising: administering a lipo-restoring composition to the face, wherein the lipo-restoring composition comprises a combination of a resorbable matrix and fat.

The method of any previous embodiment, wherein the fat is from the subject or an unrelated subject.

The method of any previous embodiment, wherein facial lipoatrophy comprises one or more of: mid-face volume deficit, mild to severe submalar volume deficit, mild to severe perioral volume deficit, or subcutaneous contour defects.

The method of any previous embodiment, wherein the defects arise from HIV infection or HAART treatment.

The method of any previous embodiment, wherein the defects are from one or more of: secondary to congenital abnormalities, trauma, surgical resection, aging processes, and disease.

The method of any previous embodiment, wherein the defects arise from infection, diabetes, auto immune disease, acquired generalized lipodystrophy (AGL), Lawrence syndrome, acquired partial lipodystrophy (APL), progressive lipodystrophy, Barraquer-Simons syndrome, injury, weight loss, repeated injection site, or localized pressure.

The method of any previous embodiment, wherein an infection comprises one or more of measles, pneumonia, infectious mononucleosis, or hepatitis.

The method of any previous embodiment, wherein the facial volume is measured against a baseline.

The method of any previous embodiment, wherein the baseline comprises a measurement made prior to administration.

The method of any previous embodiment, wherein the facial volume change is for greater than 9 months.

The method of any previous embodiment, wherein the facial volume change is for greater than 12 months.

The method of any previous embodiment, wherein the facial volume change is for greater than 18 months.

The method of any previous embodiment, wherein a female subject responds better than a male subject, wherein responding better comprises having a greater average facial volume increase at 6 months than a male.

The method of any previous embodiment, wherein a subject that does not consume alcohol responds better than a subject that does, wherein responding better comprises having a greater average facial volume increase at 6 months than a subject that does consume alcohol.

The method of any previous embodiment, wherein at 3 months a subject has from about 54.2 to about 149.5% retention of volume.

The method of any previous embodiment, wherein at 6 months a subject has from about 43.3 to about 115.6% retention of volume.

The method of any previous embodiment, wherein at 12 months after administration from about 84% to about 102% of administered is retained.

The method of any previous embodiment, wherein at one month after administration of the lipo-restoring composition, the subject has a Global Aesthetic Improvement Scale (GAIS) as measured by the subject of from about 1 to about 5; from about 2 to about 4; of about 2.8 to about 3.

The method of any previous embodiment, wherein at two months after administration of the lipo-restoring composition, the subject has a Global Aesthetic Improvement Scale (GAIS) as measured by the subject of from about 1 to about 5; from about 2 to about 4; of about 2.8 to about 3.

The method of any previous embodiment, wherein at six months after administration of the lipo-restoring composition, the subject has a Global Aesthetic Improvement Scale (GAIS) as measured by the subject of from about 1 to about 5; from about 2 to about 3; of about 2.7 to about 3.

The method of any previous embodiment, wherein at one month after administration of the lipo-restoring composition, the subject has a Global Aesthetic Improvement Scale (GAIS) as measured by blinded evaluators of from about 2 to about 4.5; from about 3 to about 4; of about 3 to about 3.3.

The method of any previous embodiment, wherein at two months after administration of the lipo-restoring composition, the subject has a Global Aesthetic Improvement Scale (GAIS) as measured by blinded evaluators of from about 2.5 to about 5; from about 3.5 to about 4.5; of about 4.

The method of any previous embodiment, wherein at six months after administration of the lipo-restoring composition, the subject has a Global Aesthetic Improvement Scale (GAIS) as measured by blinded evaluators of from about 3 to about 5 or about 4.

The method of any previous embodiment, wherein at one month after administration of the lipo-restoring composition, the subject has a Rosenberg Self Esteem Score (RSS) measured as a change from baseline of about −2.4 to −0.5; or about −1.4.

The method of any previous embodiment, wherein at three months after administration of the lipo-restoring composition, the subject has a Rosenberg Self Esteem Score (RSS) measured as a change from baseline of about −3.3 to −0.3, or about −1.8.

The method of any previous embodiment, wherein at six months after administration of the lipo-restoring composition, the subject has a Rosenberg Self Esteem Score (RSS) measured as a change from baseline of about −3.1 to −0.1, or about −1.6.

The method of any previous embodiment, wherein at one month after administration of the lipo-restoring composition, the subject has a Body Image Quality of Life Inventory (BIQLI-SP) as measured as a change from baseline of about 12.5 to about 38.0; or about 25.2.

The method of any previous embodiment, wherein at three months after administration of the lipo-restoring composition, the subject has a Body Image Quality of Life Inventory (BIQLI-SP) as measured as a change from baseline of about 3.8 to about 33.6; or about 18.7.

The method of any previous embodiment, wherein at six months after administration of the lipo-restoring composition, the subject has a Body Image Quality of Life Inventory (BIQLI-SP) as measured as a change from baseline of about 7.5 to about 36.2; or about 21.8.

The method of any previous embodiment, wherein the resorbable matrix comprises a hydrogel.

The method of any previous embodiment, wherein the hydrogel comprises thiol-modified hyaluronan, thiol-modified gelatin.

The method of any previous embodiment, wherein the hydrogel further comprising a crosslinker.

The method of any previous embodiment, wherein the crosslinkers comprise one or more of bi-, tri-, multi-functionalized molecules that are reactive to thiols, oxidation agents that initiate crosslinking, and environment influences.

The method of any previous embodiment, wherein the crosslinker comprises polyethylene glycol diacrylate.

The method of any previous embodiment, wherein the hydrogel is hyaluronan based with a hyaluronan concentration of between about 1 mg/mL to about 20 mg/mL.

The method of any previous embodiment, wherein the hydrogel is hyaluronan based with a hyaluronan concentration of between about 3 mg/mL to about 5 mg/mL.

The method of any previous embodiment, wherein the hydrogel further comprises gelatin at a concentration of between about 1 mg/mL to about 20 mg/mL.

The method of any previous embodiment, wherein the hydrogel further comprises gelatin at a concentration of between about 3 mg/mL to about 5 mg/mL.

The method of any previous embodiment, wherein the hydrogel has a weight ratio of the hyaluronan to the gelatin of between about 1:1 to about 10:1; between about 1:1 to about 1:10; about 1:1.5; about 1.5:1; about 1:2; about 2:1; or from between about 0.5:5 to about 5:0.5.

The method of any previous embodiment, wherein the thiol-modified hyaluronan has a molecular mass of at least 55000 g/mol; at least 100,000 g/mol; at least 120,000 g/mol; at least 150,000 g/mol; at least 170,000 g/mol; at least 175,000 g/mol; or at least 200,000 g.mol.

The method of any previous embodiment, wherein the thiol-modified hyaluronan comprises more than 150 μmol/g of polymer; more than 200 μmol/g of polymer; more than 1000 μmol/g of polymer; more than 10,000 μmol/g of polymer.

The method of any previous embodiment, wherein the thiol-modified collagen comprises from about 1% to about 75% of the thiol groups in the resorbable matrix.

The method of any previous embodiment, wherein the thiol-modified hyaluronan comprises from about 1% to about 75% of the thiol groups in the resorbable matrix.

The method of any previous embodiment, wherein the lipo-restoring composition is administered when the lipo-restoring composition is at about G′ 1 to about 5 Pa; or at about 0.3 to about 20 Pa; or at about 0.5 to about 10 Pa; or at about 0.75 to about 7.5 Pa.

The method of any previous embodiment, wherein the lipo-restoring composition is administered when the lipo-restoring composition is at about 1 to about 5% of its final stiffness; or about 0.1 to about 50% of its final stiffness; about 5 to about 75% of its final stiffness; or about 2 to about 4% of its final stiffness.

The resorbable matrix of any previous embodiment, further comprising phosphate salts.

The method of any previous embodiment, wherein the resorbable matrix has a storage modulus value of between about 1 Pa and 1,000 Pa.

The method of any previous embodiment, wherein the lipo-restoring composition is administered while the lipo-restoring composition has a storage modulus of between about 0.1 Pa and about 10 Pa and wherein the lipo-restoring composition continues to cure in situ to between about 20 Pa to about 150 Pa.

The method of any previous embodiment, wherein the thiol-modified hyaluronan, thiol-modified gelatin, and polyethylene glycol diacrylate (PEGDA) are in a lyophilized format.

The method of any previous embodiment, wherein the hydrogel is made by a method comprising: (a) reconstituting the thiol-modified hyaluronan, thiol-modified gelatin, and polyethylene glycol diacrylate (PEGDA); and (b) mixing the thiol-modified hyaluronan, thiol-modified gelatin, and polyethylene glycol diacrylate (PEGDA) together.

The method of any previous embodiment, wherein the hydrogel is made by a method comprising: (1) reacting a first thiolated polymer with GSSG; and (2) adding a second thiolated polymer to the reaction, thereby forming a hydrogel comprising the first and second thiolated polymers, wherein GSSG is not crosslinked to a polymer.

The method of any previous embodiment, wherein the first thiolated polymer is thiolated carboxymethylated hyaluronan and wherein the second thiolated polymer is thiolated gelatin.

The method of any previous embodiment, wherein the resorbable matrix comprises SLF.

The method of any previous embodiment, wherein SLF is made by a method comprising: (a) thawing a combination of thiol-modified hyaluronan and thiol-modified gelatin at a temperature of approximately 35° C. or greater; and (b) adding polyethylene glycol diacrylate (PEGDA) to the thawed combination of thiol-modified hyaluronan and thiol-modified gelatin.

The method of any previous embodiment, wherein the fat cells are obtained by liposuction.

The method of any previous embodiment, wherein about 50-500 mL of fat is collected from the liposuction.

The method of any previous embodiment, wherein the resorbable matrix forms a gel, and wherein the fat is mixed with the resorbable matrix before gelation is completed.

The method of any previous embodiment, wherein the resorbable matrix crosslinks before, during and/or after administration.

The method of any previous embodiment, wherein the resorbable matrix crosslinks before, during and/or after the fat is mixed with the resorbable matrix.

The method of any previous embodiment, wherein the resorbable matrix begins to crosslink before the fat is mixed with the resorbable matrix.

The method of any previous embodiment, wherein the resorbable matrix continues to crosslink after administration of the lipo-restoring composition.

The method of any previous embodiment, wherein the lipo-restoring composition is made by a method comprising: mixing the fat with the resorbable matrix, drawing the fat-resorbable matrix composition into a syringe, and allowing the resorbable matrix to gel for between about 2 to 30 minutes.

The method of any previous embodiment, wherein the syringe with the lipo-restoring composition is inverted and/or rotated several times to obtain a homogenous composition.

The method of any previous embodiment, wherein the lipo-restoring composition has a resorbable matrix to fat weight ratio of between about 1:1 to about 10:1.

The method of any previous embodiment, wherein the lipo-restoring composition is administered by injection.

The method of any previous embodiment, wherein the lipo-restoring composition is administered about 5 to about 40 minutes, about 10 to about 30 minutes or 15 to about 20 minutes post mixing of components.

The method of any previous embodiment, wherein 5 ml of the lipo-restoring composition is administered to the face.

The method of any previous embodiment, wherein between about 1 ml and about 5 ml of the lipo-restoring composition is administered to the face.

The method of any previous embodiment, wherein between about 5 ml and about 10 ml of the lipo-restoring composition is administered to the face.

The method of any previous embodiment, wherein between about 1 ml and about 20 ml of the lipo-restoring composition is administered to the face.

The method of any previous embodiment, wherein between about 1 ml and about 40 ml of the lipo-restoring composition is administered to the face.

The method of any previous embodiment, wherein between about 5 ml and about 15 ml of the lipo-restoring composition is administered to each side of the face.

The method of any previous embodiment, wherein the lipo-restoring composition comprises an implant.

A method of slowing the progression of facial lipoatrophy, comprising: administering a lipo-restoring composition to the face, wherein the lipo-restoring composition comprises a combination of a resorbable matrix and adipose derived cells or fat.

A method of preventing facial volume decrease, comprising: administering a lipo-restoring composition to the face, wherein the lipo-restoring composition comprises a combination of a resorbable matrix and adipose derived cells or fat.

A method of restoring facial volume, comprising: comprising: administering a lipo-restoring composition to the face, wherein the lipo-restoring composition comprises a combination of a resorbable matrix and adipose derived cells or fat.

A method of increasing facial volume for greater than 6 months, comprising: administering a lipo-restoring composition to the face, wherein the lipo-restoring composition comprises a combination of a resorbable matrix and adipose derived cells or fat.

A method of treating subcutaneous facial lipoatrophy defects, comprising administering a lipo-restoring composition to the face, wherein the lipo-restoring composition comprises a combination of a resorbable matrix and adipose derived cells or fat.

A stable liquid resorbable matrix comprising: thiol-modified hyaluronan and thiol-modified collagen combined in a first container with a liquid to form a stable liquid resorbable matrix.

The resorbable matrix of any previous embodiment, wherein the thiol-modified hyaluronan and thiol-modified collagen liquid is stored from between −80 degrees C. to about 45 degrees C., or from between −20 degrees C. to about 25 degrees C., from between −10 degrees C. to about 4 degrees C., or from between 0 degrees C. to about 10 degrees C.

The resorbable matrix of any previous embodiment, wherein the resorbable matrix is combined with cells or tissue to form a lipo-restoring composition.

The resorbable matrix of any previous embodiment, further comprising a crosslinker.

The resorbable matrix of any previous embodiment, wherein the crosslinkers comprise one or more of bi-, tri-, multi-functionalized molecules that are reactive to thiols, oxidation agents that initiate crosslinking, and environment influences.

The resorbable matrix of any previous embodiment, wherein the crosslinker comprises polyethylene glycol diacrylate.

The resorbable matrix of any previous embodiment, further comprising phosphate salts.

A method of, one or more of, slowing the progression of lipoatrophy, slowing the progression of facial lipoatrophy, preventing facial volume decrease, restoring facial volume, increasing facial volume for greater than 6 months, or treating subcutaneous facial lipoatrophy defects in a subject, comprising: administering a lipo-restoring composition to the face, wherein the lipo-restoring composition comprises a combination of a resorbable matrix and stromal vascular fraction (SVF), wherein the SVF contains between about 4×10⁷ to about 9×10⁷ SVF cells.

A method of, one or more of, slowing the progression of lipoatrophy, slowing the progression of facial lipoatrophy, preventing facial volume decrease, restoring facial volume, increasing facial volume for greater than 6 months, or treating subcutaneous facial lipoatrophy defects in a subject, comprising: administering a lipo-restoring composition to the face, wherein the lipo-restoring composition comprises a combination of a resorbable matrix and stromal vascular fraction (SVF), wherein the SVF contains between about 6×10⁷ and about 8×10⁷ viable SVF cells.

The method of any previous embodiment, wherein the resorbable matrix crosslinks before, during and/or after administration.

The method of any previous embodiment, wherein the resorbable matrix crosslinks before, during and/or after the SVF is mixed with the resorbable matrix.

The method of any previous embodiment, wherein the resorbable matrix begins to crosslink before the SVF is mixed with the resorbable matrix.

The method of any previous embodiment, wherein the resorbable matrix continues to crosslink after administration of the lipo-restoring composition.

The method of any previous embodiment wherein the lipo-restoring composition is administered by injection.

The method of any previous embodiment, wherein the lipo-restoring composition is administered about 5 to about 40 minutes, about 10 to about 30 minutes or 15 to about 20 minutes post mixing of components.

The method of any previous embodiment, wherein the components comprise, SVF, a thiol-modified hyaluronan and a thiol-modified collagen.

The method of any previous embodiment, wherein the components further comprise a crosslinker.

The method of any previous embodiment wherein the crosslinker comprise one or more of bi-, tri-, multi-functionalized molecules that are reactive to thiols, and/or oxidation agents that initiate crosslinking.

The method of any previous embodiment, wherein the crosslinker comprises polyethylene glycol diacrylate.

The method of any previous embodiment, wherein the thiol-modified hyaluronan has a molecular mass of at least 55000 g/mol; at least 100,000 g/mol; at least 120,000 g/mol; at least 150,000 g/mol; at least 170,000 g/mol; at least 175,000 g/mol; or at least 200,000 g/mol.

The method of any previous embodiment, wherein the thiol-modified hyaluronan comprises more than 150 μmol/g of polymer; more than 200 μmol/g of polymer; more than 1000 μmol/g of polymer; more than 10,000 μmol/g of polymer.

The method of any previous embodiment, wherein the thiol-modified collagen comprises from about 1% to about 75% of the thiol groups in the resorbable matrix.

The method of any previous embodiment, wherein the thiol-modified hyaluronan comprises from about 1% to about 75% of the thiol groups in the resorbable matrix.

The method of any previous embodiment, wherein the lipo-restoring composition is administered when the lipo-restoring composition is at about G′ 1 to about 5 Pa; or at about 0.3 to about 20 Pa; or at about 0.5 to about 10 Pa; or at about 0.75 to about 7.5 Pa.

The method of any previous embodiment, wherein the lipo-restoring composition is administered when the lipo-restoring composition is at about 1 to about 5% of its final stiffness; or about 0.1 to about 50% of its final stiffness; about 5 to about 75% of its final stiffness; or about 2 to about 4% of its final stiffness.

A method of, one or more of, slowing the progression of lipoatrophy, slowing the progression of facial lipoatrophy, preventing facial volume decrease, restoring facial volume, increasing facial volume for greater than 18 months, or treating subcutaneous facial lipoatrophy defects in a subject, comprising: administering a lipo-restoring composition to the face, wherein the lipo-restoring composition comprises a combination of a resorbable matrix and adipose derived cells.

A method of correcting moderate to severe facial wrinkles and folds, such as nasolabial folds or lip augmentation, comprising administering a lipo-restoring composition to the subcutaneous and/or supraperiosteal tissue of a subject.

A method of augmentation to correct age-related volume deficit in the mid-face comprising administering a lipo-restoring composition to the subcutaneous and/or supraperiosteal tissue of a subject.

The method of any previous embodiment, wherein the subject is over the age of 21.

The method of any previous embodiment, wherein mid-face comprises the zygomaticomalar region, anteromedial cheek, and/or submalar region.

The method of any previous embodiment, further comprising a touch-up treatment approximately 30 days after initial injection.

The method of any previous embodiment, wherein the administering is by a multi-injection technique and/or in an antegrade or retrograde fashion.

The method of any previous embodiment, wherein the multi-injection technique comprises tunneling, fanning, crosshatching, ferning, and serial puncture.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

1.-193. (canceled)

194. A method of treating a condition associated with lipoatrophy in a subject, the method comprising administering a lipo-restoring composition to the subject, wherein the lipo-restoring composition comprises a combination of a resorbable matrix and adipose derived cells.

195. The method of claim 194, which comprises: (a) restoring tissue to the lipoatrophic area, (b) treating a soft tissue defect associated with lipoatrophy, (c) slowing progression of lipoatrophy, (d) slowing progression of facial lipoatrophy, (e) preventing facial volume decrease, (f) restoring facial volume, (g) increasing facial volume for greater than 6 months, (h) treating a subcutaneous facial lipoatrophy defect, or (i) any combination thereof, in a subject.

196. The method of claim 195, wherein the defect is associated with HIV infection, HAART treatment, congenital abnormalities, trauma, surgical resection, aging processes, disease, or any combination thereof.

197. The method of claim 194, wherein the adipose derived cells comprise one or more of: autologous adipose derived cells, stromal vascular cells, stromal vascular fraction, multipotent stem cells, pre-adipocytes, and endothelial precursor cells.

198. The method of claim 194, wherein the adipose derived cells comprise fat tissue cells of the subject.

199. The method of claim 194, wherein the resorbable matrix comprises a hydrogel.

200. The method of claim 199, wherein the hydrogel comprises thiol-modified hyaluronan, thiol-modified gelatin, and polyethylenegycol diacrylate (PEGDA).

201. The method of claim 200, wherein the thiol-modified hyaluronan, thiol-modified gelatin, and polyethylenegycol diacrylate (PEGDA) are lyophilized.

202. The method of claim 199, wherein the hydrogel comprises hyaluronan at a concentration between about 1 mg/mL to about 20 mg/mL.

203. The method of claim 202, wherein the hydrogel further comprises gelatin at a concentration between about 1 mg/mL to about 20 mg/mL.

204. The method of claim 194, wherein the resorbable matrix comprises SLF.

205. The method of claim 194, wherein the lipo-restoring composition comprises the adipose derived cells suspended in the resorbable matrix.

206. The method of claim 194, wherein about 5 ml of the lipo-restoring composition is administered to the face.

207. The method of claim 194, wherein about 1 ml to about 40 ml of the lipo-restoring composition is administered to the face.

208. A stable liquid resorbable matrix comprising thiol-modified hyaluronan and thiol-modified collagen in a liquid.

209. The resorbable matrix of claim 208, wherein the thiol-modified hyaluronan and thiol-modified collagen liquid is stored at a temperature between about −80 degrees C. to about 45 degrees C., about −20 degrees C. to about 25 degrees C., about −10 degrees C. to about 4 degrees C., or about 0 degrees C. to about 10 degrees C.

210. The resorbable matrix of claim 208, wherein the resorbable matrix is combined with cells or tissue to form a lipo-restoring composition.

211. The resorbable matrix of claim 208, further comprising a crosslinker.

212. The resorbable matrix of claim 211, wherein the crosslinker comprises one or more of bi-, tri-, multi-functionalized molecules that are reactive to thiols, oxidation agents that initiate crosslinking, and environment influences.

213. The resorbable matrix of claim 211, wherein the crosslinker comprises polyethylene glycol diacrylate.

214. A method of slowing the progression of lipoatrophy, slowing the progression of facial lipoatrophy, preventing facial volume decrease, restoring facial volume, increasing facial volume for greater than 6 months, treating subcutaneous facial lipoatrophy defects, or any combination thereof, in a subject, the method comprising administering a lipo-restoring composition to the face, wherein the lipo-restoring composition comprises a resorbable matrix and a stromal vascular fraction (SVF), wherein the SVF comprises between about 4×10⁷ to about 9×10⁷ SVF cells.
